(19) United States
(12) Patent Application Publication
(10) Pub. No.: US 2025/0281059 A1
(43) Pub. Date: Sep. 11, 2025

(54) MONITORING A HEMODYNAMIC STATUS OF A PATIENT

(71) Applicant: Masimo Corporation, Irvine, CA (US)

(72) Inventor: Alberto Avendano, Anaheim, CA (US)

(21) Appl. No.: 19/060,504

(22) Filed: Feb. 21, 2025

Related U.S. Application Data
(60) Provisional application No. 63/562,615, filed on Mar. 7, 2024.

(51) Int. Cl. A61B 5/029; A61B 5/00; A61B 50/20; G16H 40/63

(52) U.S. Cl. CPC A61B 5/029; A61B 5/7435; A61B 5/7475; A61B 50/20; G16H 40/63

(57) ABSTRACT

A system for monitoring hemodynamic status of a patient can include a transducer, an adapter and one or more monitor devices. The adapter may be in communication with the transducer and the one or more monitor devices. The adapter can be configured to receive and process data from the transducer such as unprocessed physiological data. The adapter can be configured to transmit data to the monitor device(s) such as processed and/or unprocessed physiological data. The adapter can be configured to generate, and transmit to the monitor devices(s), user interface data for rendering interactive graphical user interfaces to display information such as physiological information relating to a hemodynamic status of the patient. The adapter can be configured to receive and process, from the monitor device(s) user commands or instructions to control an operation of the system or its components.

### INCORPORATION BY REFERENCE TO ANY PRIORITY APPLICATIONS

This application claims priority to U.S. Provisional Application No. 63/562,615, entitled “SYSTEM AND DEVICES FOR MONITORING A HEMODYNAMIC STATUS OF A PATIENT,” filed on Mar. 7, 2024, the entire contents of which are hereby incorporated herein by reference in their entirety.

Any and all applications for which a foreign or domestic priority claim is identified in the Application Data Sheet as filed with the present application are hereby incorporated herein by reference under 37 CFR 1.57.

### TECHNICAL FIELD

The present disclosure relates to devices, methods, and/or systems for monitoring a patient's hemodynamic status and/or cardiac output.

### BACKGROUND

An accurate knowledge of the hemodynamic status/cardiac output of the heart of a patient helps medical practitioners assess a patient's medical condition. The constituents of cardiac output (typically measured, for example, in liters/minute), heart rate (typically measured, for example, in beats per minute) and stroke volume (typically measured, for example, in mls) may also provide useful information. The stroke volume, or cardiac stroke volume, is the volume of blood ejected by the left ventricle during systole across the aortic valve forwards into the aorta during each cardiac contraction. This volume normally corresponds to the volume of blood in the left ventricle at the end of the systole minus the pre-systole diastolic volume of the left ventricle. This is particularly true in acute situations, such as, for example, for patients in intensive care units or patients undergoing an operation where for example it is used in fluid and drug management during anesthesia and after. Knowledge of a patient's cardiac output, or its constituents, may, moreover, be beneficial in less critical or less life-threatening situations, such as in situations where the monitoring of the patient is generally desirable.

### SUMMARY

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

In some aspects, the techniques described herein relate to a system for monitoring a hemodynamic status of a patient, the system including: a transducer configured to couple to the patient and measure a hemodynamic status of the patient; a holder configured to couple the transducer to a mounting plate, the holder comprising: a first movable arm configured to engage a first bracket arm of the mounting plate, the first movable arm including a first securing device extending from the first movable arm; and an adapter in communication with the transducer and a display and comprising one or more hardware processors, wherein the one or more hardware processors are configured to: generate one or more signals in response to the measurement of the transducer; process the one or more signals to generate one or more physiological parameters; generate, based at least in part on the one or more signals or the one or more physiological parameters, user interface data for rendering a graphical user interface without additional signal processing by a monitor; transmit, to the monitor, the user interface data; transmit, to the monitor, the one or more signals or the one or more physiological parameters; receive, from the monitor, user input; and execute instructions to control an operation of the adapter or transducer or monitor, according to the user input.

In some aspects, the techniques described herein relate to a system, wherein the system comprises a second movable arm configured to engage a second bracket arm of the mounting plate, the second movable arm including a second securing device extending from the second movable arm.

In some aspects, the techniques described herein relate to a system, wherein the hemodynamic status includes a cardiac output of the patient.

In some aspects, the techniques described herein relate to a system, wherein the transducer uses a lithium chloride indicator method to measure the hemodynamic status of the patient.

In some aspects, the techniques described herein relate to a system, wherein the one or more hardware processors are configured to not transmit, to the monitor, the one or more signals, and to transmit, to the monitor, the one or more physiological parameters.

In some aspects, the techniques described herein relate to a system, wherein the adapter includes a button configured to cause the one or more hardware processors to alter a value of the one or more physiological parameters to an altered value.

In some aspects, the techniques described herein relate to a system, wherein the altered value includes a default value or zero value.

In some aspects, the techniques described herein relate to a system, wherein the transducer is configured to communicate with a patient monitor through an adapter.

In some aspects, the techniques described herein relate to a system, wherein the holder is configured to secure to a plurality of mounting plates with varying depths of the first bracket arm and the second bracket arm.

In some aspects, the techniques described herein relate to a system, wherein the first movable arm or the second movable arm is configured to be depressed inwards towards the transducer.

In some aspects, the techniques described herein relate to a system, wherein the first movable arm or the second movable arm is configured to provide an outward force towards the first bracket arm or the second bracket arm when depressed inwards.

In some aspects, the techniques described herein relate to a system, wherein the first movable arm or the second movable arm are integrated into the holder.

In some aspects, the techniques described herein relate to a system, wherein the first movable arm or the second movable arm is cutout of a portion of the holder.

In some aspects, the techniques described herein relate to a system, wherein the first securing device or the second securing device is cutout of a portion of the holder.

In some aspects, the techniques described herein relate to a system, wherein the first securing device or the second securing device is integrated into the holder.

In some aspects, the techniques described herein relate to a system, wherein the first securing device or the second securing device is configured to be depressed inwards towards the transducer.

In some aspects, the techniques described herein relate to a system, wherein the first securing device or the second securing device is configured to provide an outward force towards the first bracket arm or the second bracket arm when depressed inwards.

In some aspects, the techniques described herein relate to a system, wherein the first bracket arm is separated from the second bracket arm by a width.

In some aspects, the techniques described herein relate to a system, wherein the width includes a distance between and including 26 mm and 30 mm.

In some aspects, the techniques described herein relate to a system, wherein the first movable arm or the second movable arm are integrated into the holder.

In some aspects, the techniques described herein relate to a system, wherein the first movable arm or the second movable arm are cutout of a portion of the holder.

In some aspects, the techniques described herein relate to a system, wherein the system further comprises the monitor, the monitor comprising the display and configured to display graphical user interfaces via the display and receive user input via the display.

In some aspects, the techniques described herein relate to a system, wherein the holder is configured to couple the transducer to the mounting plate without using any adhesive.

In some aspects, the techniques described herein relate to a system for monitoring a hemodynamic status of a patient, the system including: a holder configured to couple a transducer to a mounting plate, wherein the transducer is configured to couple to a patient and measure a hemodynamic status of the patient, the holder comprising: a first movable arm configured to engage a first bracket arm of the mounting plate, the first movable arm including a first securing device extending from the first movable arm; and a second movable arm configured to engage a second bracket arm of the mounting plate, the second movable arm including a second securing device extending from the second movable arm; and wherein the holder is configured to secure to a plurality of mounting plates with varying widths.

In some aspects, the techniques described herein relate to a system, wherein the transducer is configured to communicate with a patient monitor through an adapter.

In some aspects, the techniques described herein relate to a system, wherein the holder is configured to secure to a plurality of mounting plates with varying depths of the first bracket arm and the second bracket arm.

In some aspects, the techniques described herein relate to a system, wherein the first movable arm or the second movable arm is configured to be depressed inwards towards the transducer.

In some aspects, the techniques described herein relate to a system, wherein the first movable arm or the second movable arm is configured to provide an outward force towards first bracket arm or the second bracket arm when depressed inwards.

In some aspects, the techniques described herein relate to a system, wherein the first movable arm or the second movable arm are integrated into the holder.

In some aspects, the techniques described herein relate to a system, wherein the first movable arm or the second movable arm is cutout of a portion of the holder.

In some aspects, the techniques described herein relate to a system, wherein the first securing device or the second securing device is cutout of a portion of the holder.

In some aspects, the techniques described herein relate to a system, wherein the first securing device or the second securing device is integrated into the holder.

In some aspects, the techniques described herein relate to a system, wherein the first securing device or the second securing device is configured to be depressed inwards towards the transducer.

In some aspects, the techniques described herein relate to a system, wherein the first securing device or the second securing device is configured to provide an outward force towards the first bracket arm or the second bracket arm when depressed inwards.

In some aspects, the techniques described herein relate to a system, wherein the first bracket arm is separated from the second bracket arm by a width.

In some aspects, the techniques described herein relate to a system, wherein the width includes a distance between and including 26 mm and 30 mm.

In some aspects, the techniques described herein relate to a system, wherein the holder is configured to couple the transducer to the mounting plate without using any adhesive.

In some aspects, the techniques described herein relate to a system or device as described herein.

Any of the aspects or examples disclosed herein may be combined in whole or in part.

### BRIEF DESCRIPTION OF THE DRAWINGS

Certain features of this disclosure are described below with reference to the drawings. The illustrated embodiments are intended to illustrate, but not to limit, the embodiments. Various features of the different disclosed embodiments can be combined to form further embodiments, which are part of this disclosure.

FIG. 1 is a schematic block diagram illustrating an example system and devices for monitoring a patient's hemodynamic status.

FIG. 2 illustrates an example implementation of a system and devices for monitoring a patient's hemodynamic status.

FIG. 3A-3B illustrate example views of a connector and clip system of the example system of FIGS. 1 and/or 2.

FIGS. 4A-4C illustrate example views of the example connector illustrated in FIGS. 3A-3B.

FIGS. 5A-5D illustrate example views of the example clip of FIGS. 3A-3B.

FIGS. 6A-6H and 7A-7G illustrate aspects of an example transducer and transducer shroud as illustrated in FIGS. 1 and/or 2.

FIGS. 8A-8L, 9A-9G, 10A-10D, 11A-11D, 12A-12C, and 13A-13F illustrate example user interfaces that may be displayed by a monitor device of the system described herein.

FIG. 14 is a block diagram of an example computer system useable with the various implementations of the present disclosure.

### DETAILED DESCRIPTION

Various embodiments will be described hereinafter with reference to the accompanying drawings. The drawings and the associated descriptions are provided to illustrate embodiments of the present disclosure and do not limit the scope of the claims. In the drawings, similar elements have similar reference numerals.

### Example Overview of Systems and Devices

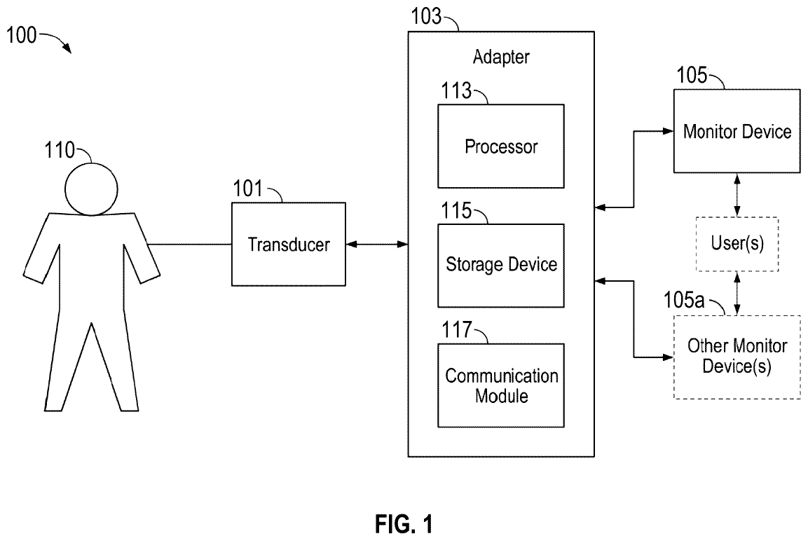

FIG. 1 is a schematic block diagram illustrating an example system 100 and devices for monitoring a patient's hemodynamic status. The system 100 can include a transducer 101, an adapter 103, and one or more monitoring devices 105. The transducer 101 may be configured to monitor a hemodynamic status of the patient 110 such as cardiac output. The transducer 101 may be configured to connect to a patient 110. For example, the transducer 101 may be coupled to tubing, catheter, cannula, or the like coupled to a patient. The transducer 101 may be in wired or wireless communication with the adapter 103. The adapter 103 may be in wired or wireless communication with the one or more monitor devices 105.

The system 100 may include a transducer 101 which may be configured to detect a hemodynamic status of the patient and, in response, generate one or more signals. The transducer 101 may be configured to generate one or more signals indicating a patient's hemodynamic status. The transducer 101 may generate and/or transmit the one or more signals indicating the patient's hemodynamic status continuously, discretely in real-time, or on a delay with the patient's actual hemodynamic status. The transducer 101 may be configured to implement one or more methods to measure a hemodynamic status. In some examples, a transducer 101 may be configured to implement a bolus indicator dilution method and/or a lithium chloride indicator dilution method to measure hemodynamic status. The transducer 101 may additionally or alternatively be configured to measure physiological parameters, such as cardiac output and/or the like.

In an example method, transducer 101 may be configured to inject and/or cause to inject, via a central or peripheral venous cannula, a bolus of lithium chloride to the patient 110. The transducer 101 may be configured to measure a resulting arterial lithium concentration time curve of the patient, for example, by withdrawing blood past a lithium sensor which may be attached to the patient's existing arterial line. In response, the transducer 101 may be configured to generate one or more signals indicating the hemodynamic status of the patient 110.

The system 100 may include an adapter 103. The adapter 103 can include at least one hardware processor 113, a storage device 115, and/or a communication module 117. The processor(s) 113 can be configured, among other things, to process data, execute instructions to perform one or more functions, and/or control the operation of the transducer 101 and/or monitor devices 105. For example, the processor(s) 113 can be configured to process physiological data obtained from the transducer 101 and/or other physiological sensors or sources of measured and/or stored physiological data. Advantageously, the processor(s) 113 may be configured to independently perform one or more signal processing instructions without additional processing from a monitor and/or external processor(s). The processor(s) 113 may be configured to output physiological data for receipt by a variety of different types of monitors. Thus, the adapter 103 may be connected to a plurality of different types of monitors, thus simplifying use of the system described herein in a setting where different types of monitors or different manufacturer monitors may be in use. Similarly, physiological data may be packaged so as to connect to a variety of types of monitors. The processor(s) 113 may be configured to generate and display, in some examples, aspects of a user interface, such as described herein. The processor(s) 113 can be configured to execute instructions to perform functions related to the physiological data. For example, the processor(s) 113 can be configured to execute instructions to perform functions related to storing and/or transmitting such physiological data, for example to the monitor device 105. In some further examples, the processor(s) 113 can be configured to execute instructions associated with one or more graphical user interfaces. For example, the processor(s) 113 can be configured to generate data for rendering graphical user interfaces, to transmit to the monitor device(s) 105 for the monitor device(s) 105, and/or to display graphical user interfaces that may be useful for monitoring a hemodynamic status of a patient. In some examples, the adapter 103 may be configured to analyze a plurality of physiological data, including but not limited to non-invasive and minimally invasive physiological sensors. Thus, the adapter may simplify a connection to a monitoring system of a number of physiological sensor types and reduce the need to have multiple sensors with multiple connection points to a patient monitor. In some examples, the processor(s) 113 may be included on board and/or internal to the adapter 103 and/or external to the adapter 103. For example, the adapter 103 may be connected wirelessly and/or via wires to an external processor 113 configured to perform some or all of the processing described herein.

The one or more storage devices 115 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can, for example, include processed and/or unprocessed physiological data obtained from the transducer 101.

The communication module 117 can facilitate communication (via wired and/or wireless connection) between the adapter 103 (and/or components thereof) and separate devices, such as the transducer 101 and/or monitoring device(s) 105. For example, the communication module 117 can be configured to allow the adapter 103 to wirelessly, and/or via a wired connection, communicate with other devices, systems, and/or networks over any of a variety of communication protocols. The communication module 117 can be configured to use any of a variety of wired or wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 117 can allow data and/or instructions to be transmitted and/or received to and/or from the adapter 103 and separate devices such as the transducer 101 and/or monitoring device(s) 105. The communication module 117 can be configured to transmit (for example, wirelessly) processed physiological data (such as physiological parameters) and/or unprocessed physiological data (such as raw signals) or other information to the monitor device(s) 105 or to separate computing devices, which can include, among others, a mobile device (for example, an iOS or Android enabled smartphone, tablet, laptop), a desktop computer, a server or other computing or processing device for display and/or further processing, among other things. As another example, the communication module 117 of the adapter 103 can be configured to wirelessly transmit processed and/or unprocessed obtained physiological information and/or other information (for example, motion and/or location data) to a mobile phone which can include one or more hardware processors configured to execute an application that generates a graphical user interface displaying information representative of the processed or unprocessed physiological and/or other information obtained from the adapter 103. The communication module 117 can be embodied in one or more components that are in communication with each other. The communication module 117 can include a wireless transceiver, an antenna, and/or a near field communication (NFC) component, for example, NFC transponder.

The system 100 may include one or more monitor devices 105. A monitor device 105 may be a physiological monitor device for monitoring a patient and/or a patient's physiological data and/or parameters. A monitor device 105 may include a display configured to display interactive graphical user interfaces. A monitor device 105 may receive processed and/or unprocessed physiological data or other information from the adapter 103 (for example, via the communication module 117). The monitor device 105 may additionally and/or alternatively receive user interface data for rendering a graphical user interface from the adapter 103. A monitor device 105 may generate interactive user interfaces. The interactive user interface(s) may be generated according to the data and/or information received from the adapter 103, for displaying on a display of the monitor device 105.

The monitor device(s) 105 may be native to the system 100 and/or may be natively compatible with the adapter 103 for example the monitor device 105 and the adapter 103 may be manufactured by the same manufacturer and/or include the same operational settings, parameters, specifications, and/or the like. In some embodiments, the monitor device(s) 105 may not be native to the system 100, for example, other monitor device(s) 105a and/or adapter 103 may be manufactured by different manufacturers and/or include different operational settings, parameters, specifications, and/or the like. In some embodiments, the communication module 117 may be configured to communicate with other monitoring device(s) 105a (e.g., non-native and/or third-party monitor devices) to allow the system 100 to operate and function as if the monitor device(s) 105a were native and as described herein.

In some embodiments, a user may interact with the monitor device(s) 105 (and/or monitor device(s) 105a, and/or the like), for example, via an interactive user interface on a display of the monitor device 105. A user may control, via the monitor device 105, the operation or functionality of the system 100 or its components and devices such as the transducer 101, the adapter 103, and/or the monitor device(s) 105. As discussed above, the adapter 103 may be configured to receive user commands or instructions (e.g., via the communication module 117) and may process said user commands or instructions (e.g., by the processor 113) and may accordingly execute instructions to perform one or more functions, and/or control the operation of the transducer 101 and/or monitor devices 105.

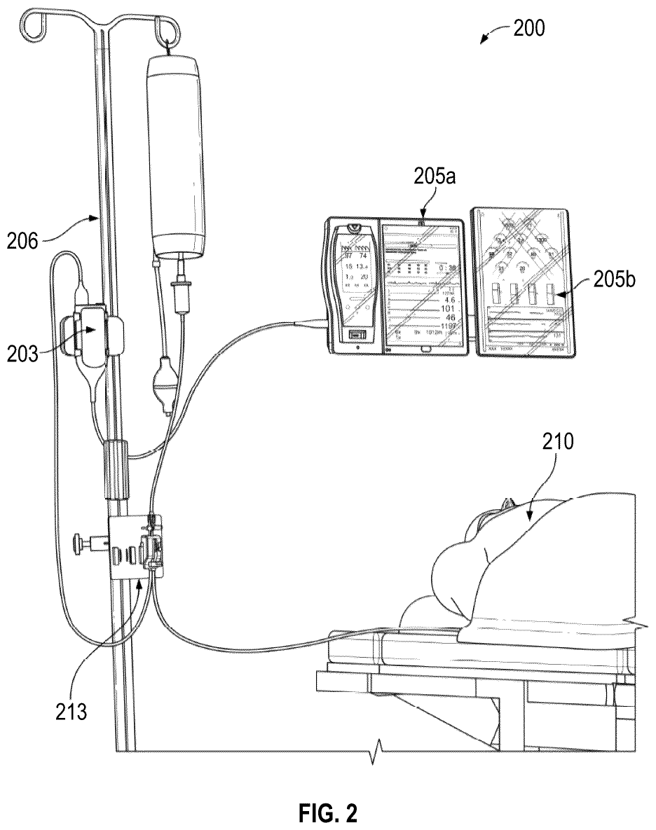

FIG. 2 illustrates an example implementation of a system 200 for monitoring a patient's hemodynamic status. The system 200 and its various devices and components may include similar structural and/or operational features as described with reference to FIG. 1. The system 200 can include a transducer 213 coupled to a patient 210 and configured to detect a patient's hemodynamic status. The system 200 can also include an adapter 203 configured to be in communication with the transducer 213 as well as one or more patient monitor devices 205a, 205b configured to be in communication with the adapter 203 and/or configured to monitor a patient 210. The adapter 203 and/or the transducer 213 may be coupled to a medical IV pole 206. The components may be configured and/or operate as discussed above with reference to FIG. 1.

### Example Aspects Related to Various Adapter and Adaptor Connector Implementations

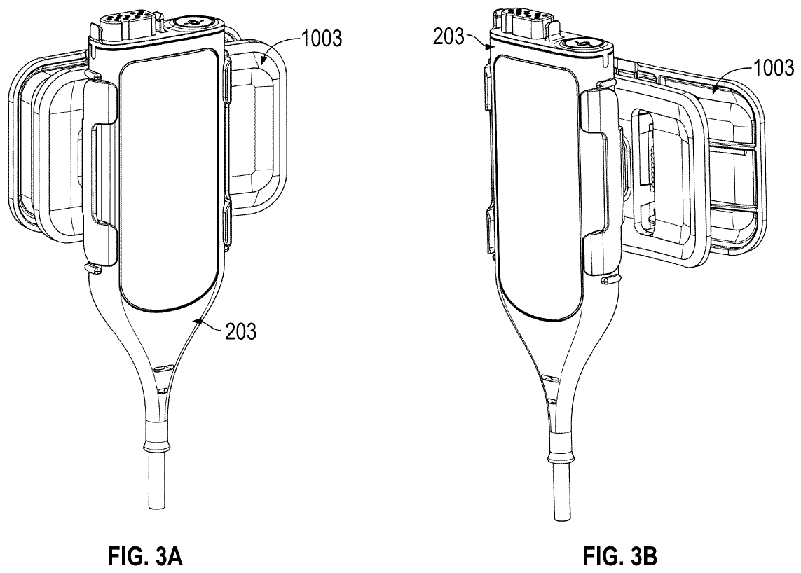

FIGS. 3A-3B, 4A-4C, and 5A-5D illustrate aspects of an example adapter and adaptor connector and/or holder that may be part of a system 100 as described in FIG. 1 and/or a system 200 as described in FIG. 2.

As illustrated in FIGS. 3A-3B, an adapter 203 may be configured to mount and/or couple to a location in a patient environment, such as a medical IV pole 206 as shown in FIG. 2, directly or indirectly. The adapter 203 may be configured to couple to a connector clip 1003. The connector clip 1003 may be configured to couple to a location in the environment of the patient, such as, for example, a medical IV pole 206 and/or another location adjacent to a patient. The adapter 203 may include one or more hardware processors configured to perform one or more processes associated with at least one physiological parameter (e.g., processor(s) 113 as described with reference to FIG. 1. For example, the one or more hardware processors may be configured to process at least one physiological parameter measured by a transducer 213 as illustrated in FIG. 2.

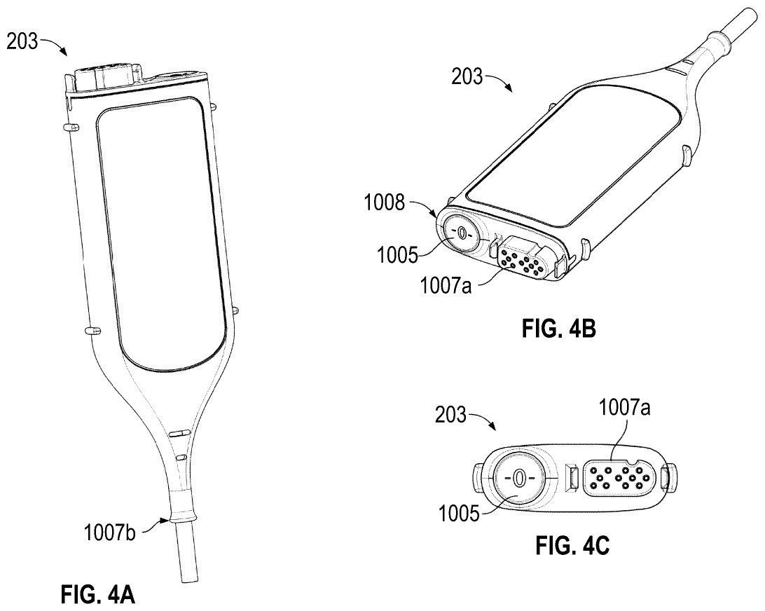

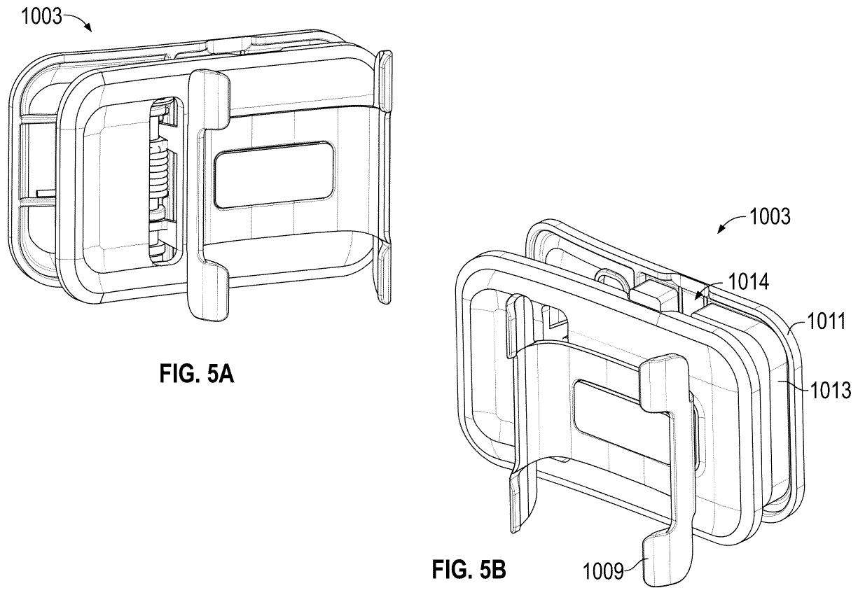

With reference to FIGS. 4A-4C, an adapter 203 may be configured to be in electronic communication with the transducer 213 and/or a patient monitor (e.g., patient monitor devices 205a and/or 205b of FIG. 2. In some examples, the adapter 203 may include at least one wired connection and/or connector 1007a configured to facilitate communication with the transducer 213 and/or adapter 203. In some examples, the adapter 203 may additionally and/or alternatively include a wireless connection to the transducer 213 and/or patient monitor devices 205a and/or 205b via for example, communication module 117 of FIG. 1. In some examples, a wired connector 1007b may be a coupled and/or attached cable. The cable may be configured to connect to a transducer 213 and/or another medical device. Additionally, in some examples, the wired connector 1007b may be a connection port for coupling the adapter 203 to a cable or other connective component, such as a wired or wireless connection. A connection port may be configured to connect to a patient monitor devices 205a and/or 205b of FIG. 2, and/or a variety of patient monitors. In the illustrated example, the adapter 203 includes a first connector 1007a and a second connector 1007b. The first connector 1007a may be a connection port configured to couple to a cable. The first connector 1007a may be configured to couple to an output and/or input. In some examples, the first connector 1007a may couple to a patient monitor through a connection cable. The first connector 1007a may be proprietary and/or unique to a patient monitor and/or a universal connector and capable for connecting to a plurality of different types of outputs or inputs. In some examples, an adapter 203 may include more than one first connector 1007a. A second connector 1007b may include a permanent or semi-permanent cable connection. For example, the second connector 1007b and associated cable connection may be configured to connect to a medical device, such as a transducer 213. In some examples, the adapter 203 may include more than two connectors 1007a and/or 1007b.

With continued reference to FIGS. 4A-4C, in some examples, an adapter 203 may include a button 1005. The button 1005 may be configured to calibrate, reset, and/or otherwise manipulate actions of the transducer 213 and/or other physiological sensors, and/or cause a controller to manipulate data associated with the transducer 213 and/or other physiological sensors. The button 1005 may be configured to be on a top surface 1008 of the adapter 203. A top surface 1008 may be a minor surface associated with a first connector 1007a and/or second connector 1007b. The location of the button 1005 may be ergonomically comfortable for a user to reach and use. The button 1005 may be located so as to reduce potential accidental engagement from the user. For example, a user may be less likely to accidentally engage the button 1005 located on a minor surface of the adapter 203 than if the button 1005 were located on a major surface (e.g., an outward and/or along the sides of the adapter 203. Hence, locating the button 1005 on a minor surface may result in more intentional engagement by a user than at another location. This minor surface can be the minor surface of the connector 1007b; thus, the button can be located next to and on the same side as the connectors 1007b.

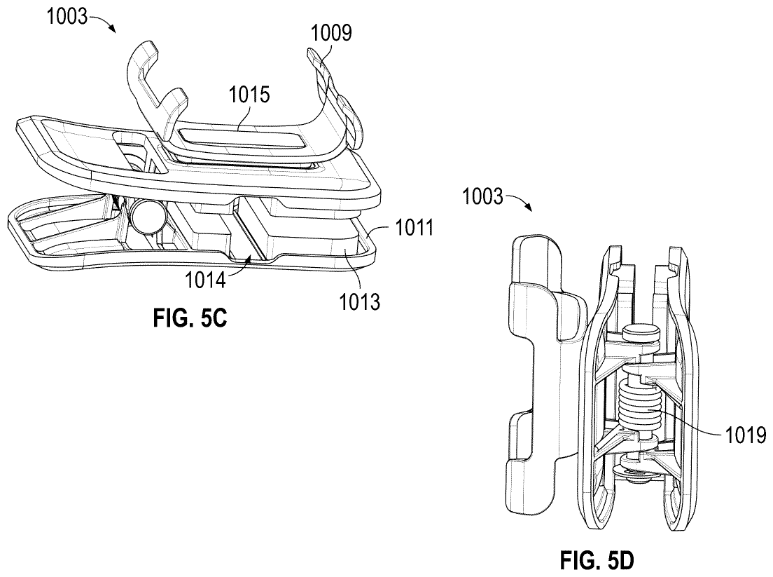

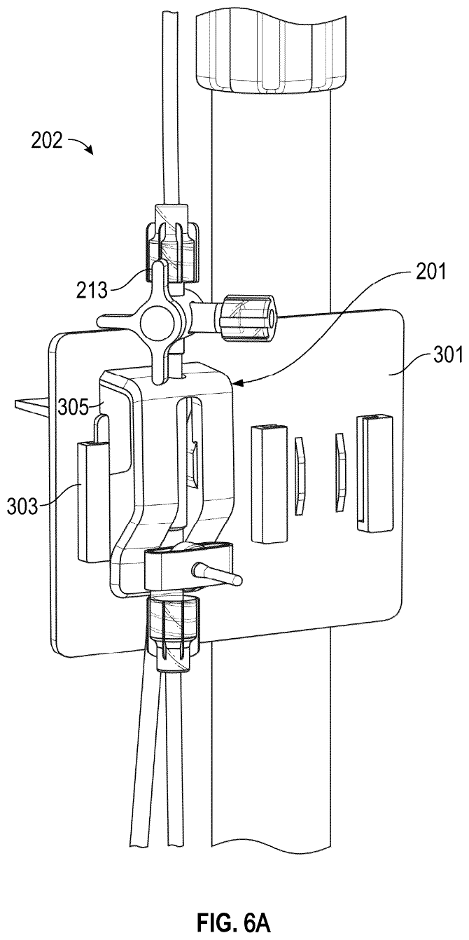

FIG. 5A-5D illustrate views of an example connector clip 1003. The connector clip 1003 is configured to couple an adapter 203 to a location in the environment of a patient. The connector clip 1003 may be configured to have a connector coupling section 1009. The connector coupling section 1009 may be configured to receive and/or hold an adapter 203. The connector coupling section 1009 may, for example, have a shape configured to conform to at least a portion of the adapter 203 so that the adapter 203 may fit within the shape. In some examples, the connector coupling section 1009 may function as a clip, snap, or other fastener configured to removably hold the adapter 203 in place on the clip 1003. In some examples, the shape may be configured to apply a sufficient force to the adapter 203, so as to reduce and/or limit unintentional vertical and/or horizontal movement of the adapter 203. In some examples, a connector clip 1003 may include a material 1015 configured to make contact with an adapter 203 when coupled to a connector clip 1003. The material 1015 may be configured to facilitate a friction force, coupling the connector clip 1003 and the adapter 203. The material 1015 may be configured to reduce or limit unintentional vertical and/or horizontal movement of the adapter 203 when coupled to the connector clip 1003. In some examples, the material 1015 may be a different material and/or include different surface features than associated components of a connector clip 1003 (e.g., such as rubber, a texturized surface, and/or the like).

The connector clip 1003 may be an alligator clip and/or other type of spring-loaded clip. However, other coupling mechanisms configured to couple an adapter to a location in the environment of a patient may additionally or alternatively be used, such as adhesive, clasp, magnetic connection, and/or the like, and/or a combination thereof. The connector clip 1003 may include one or more separate components 1011 configured to close based on a tension from a spring 1019 around a pivot point 1017. An interior portion of the components 1011 may include one or more surfaces 1013. In some examples, the one or more surfaces 1013 may be textured so as to increase a frictional force between the connector clip 1003 and a component placed between the components 1101 of the connector clip 1003, such as a medical IV pole 206 of FIG. 2. In some examples, the one or more surfaces 1013 may be padded, rubberized, and/or another material different from one or more components of the connector clip 1003. In some examples, the one or more surfaces 1013 may be a plurality of surfaces 1013 on each of the one or more components 1011. For example, one or more of the components 1011 may have two or more surfaces 1013. The two or more surfaces 1013 may have a gap 1014. The gap 1014 may be configured to allow for a medical IV pole 206 to be received between the two or more surfaces 1013. In some examples, the one or more surfaces 1013 may be indented to receive a medical IV pole 206. In some examples, the one or more surfaces 1013 on one component 1011 may be approximately parallel in whole or in part with one or more surfaces 1013 on an opposing component 1011 of the connector clip 1003.

### Example Aspects Related to Various Transducer Shroud Implementations

Embodiments described herein provide improvements over the prior art. Advantageously, the various locking features described herein, alone and/or in combination with each other, can provide mechanical coupling, alignment, and/or support mechanisms to secure a transducer shroud in substitution for applying an adhesive substance (e.g., glue and/or the like). When secured, the locking features may prevent unintentional separation of the shroud without forceful breaking of the shroud. For example, the locking features may sufficiently secure a back, middle, and/or cover component of the shroud to one another such that the locking features prevent disassembly of the shroud during use. Moreover, the locking features may increase the speed and case with which the shroud is assembled. For example, the locking features may immediately provide coupling, alignment, and/or support functionalities without a need to wait for an adhesive to dry and/or without a need to maintain components in alignment during a drying time.

Advantageously, the locking features can improve (e.g., increase) the mechanical strength of the shroud (e.g., particularly if experiencing tensile or shear forces), such that the shroud can have increased robustness against stress, vibration, and/or movement induced by various environmental factors (e.g., motion of a patient and/or medical practitioner, forces imparted during installation of the shroud, etc.) over that of a shroud secured via adhesives. For example, the locking features can help ensure that the back, middle, and/or cover components of the shroud are properly aligned during assembly of the shroud and/or remain aligned when secured to one another. The locking features can provide multiple points, lines, and/or planes of alignment to help maintain alignment of the components when secured. This can reduce lift and/or angular moment of the back, middle, and/or cover components, which may otherwise reduce the structural integrity of the shroud over time as the shroud is used. Moreover, the locking features can support a transducer while held by the shroud such that the transducer is secured and remains in a fixed position.

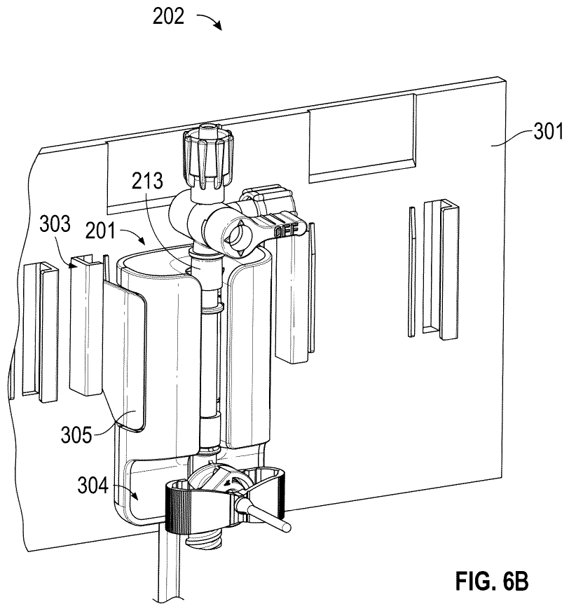

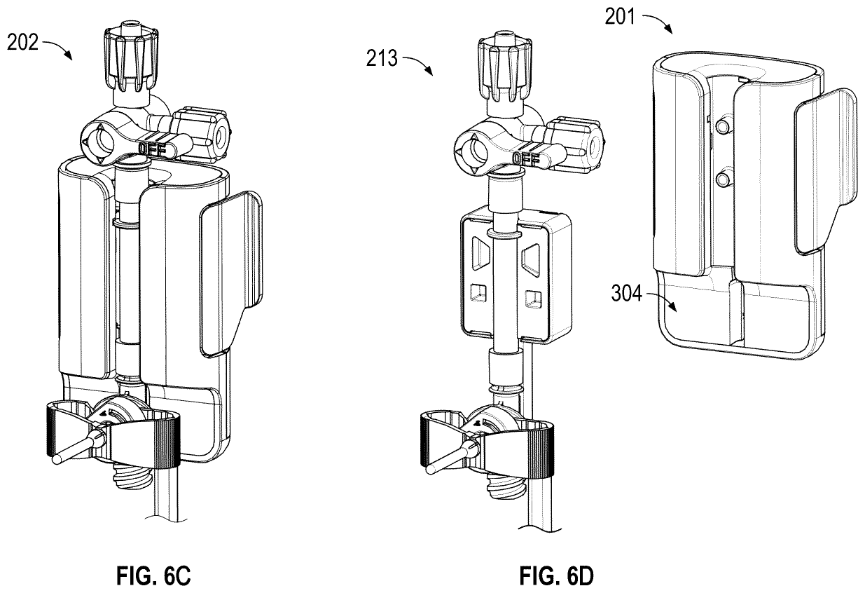

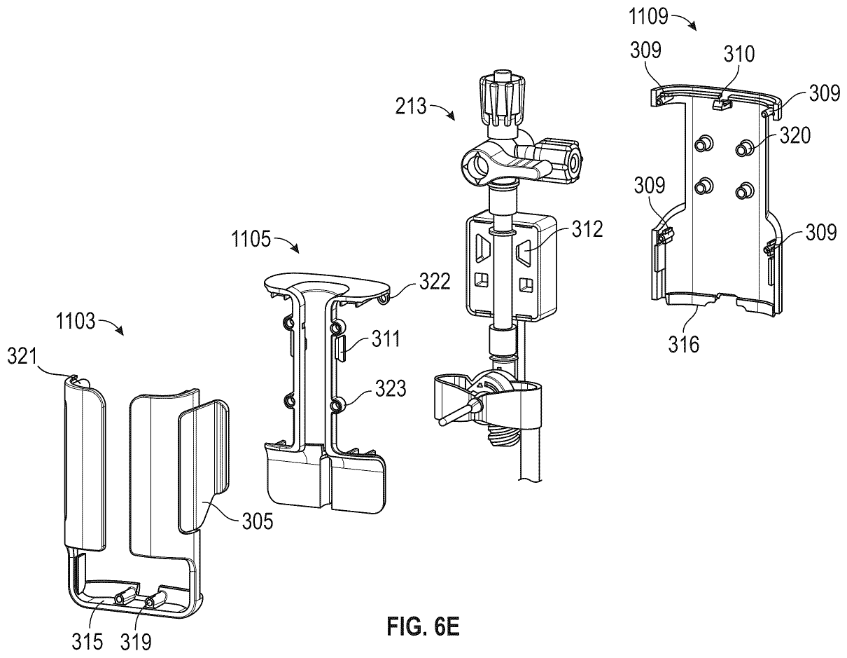

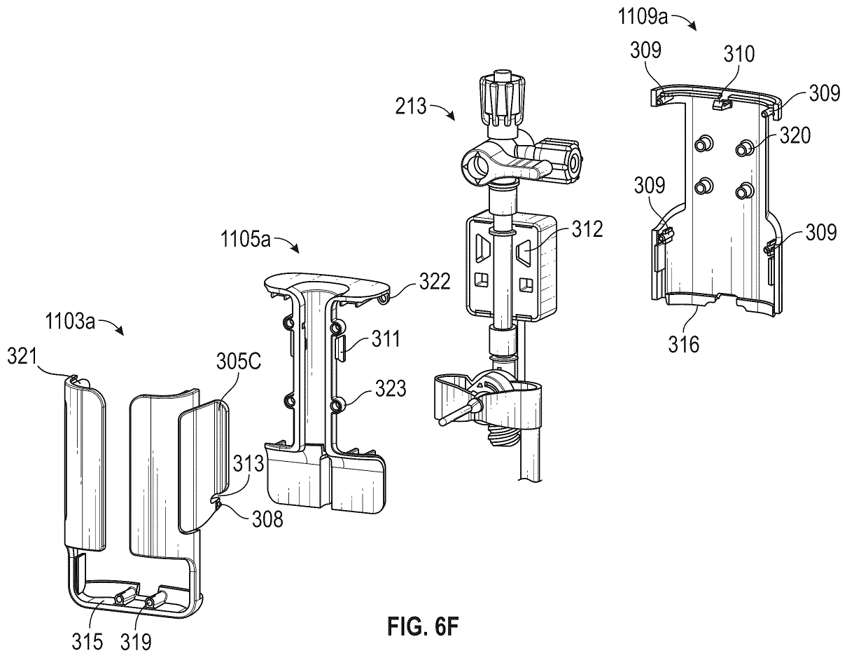

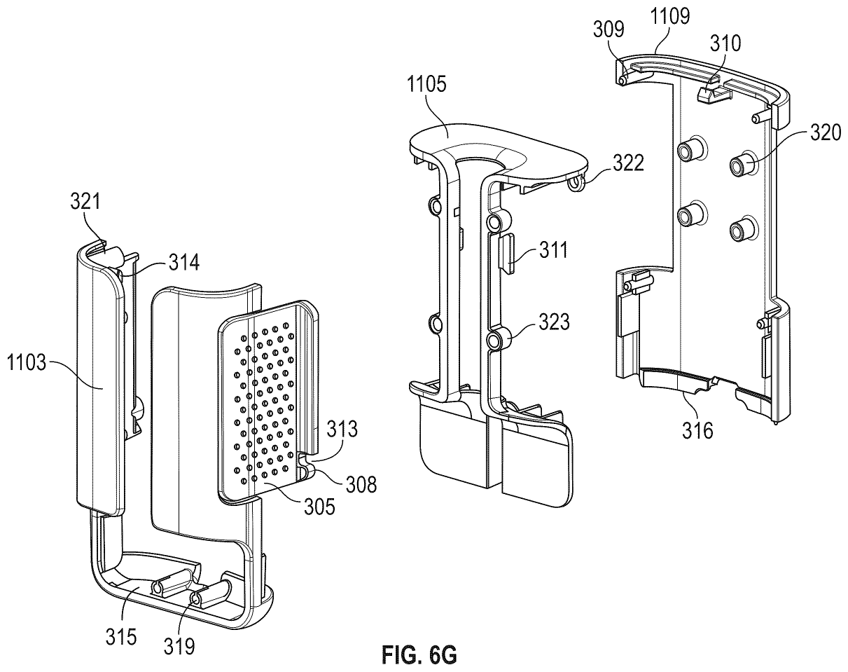

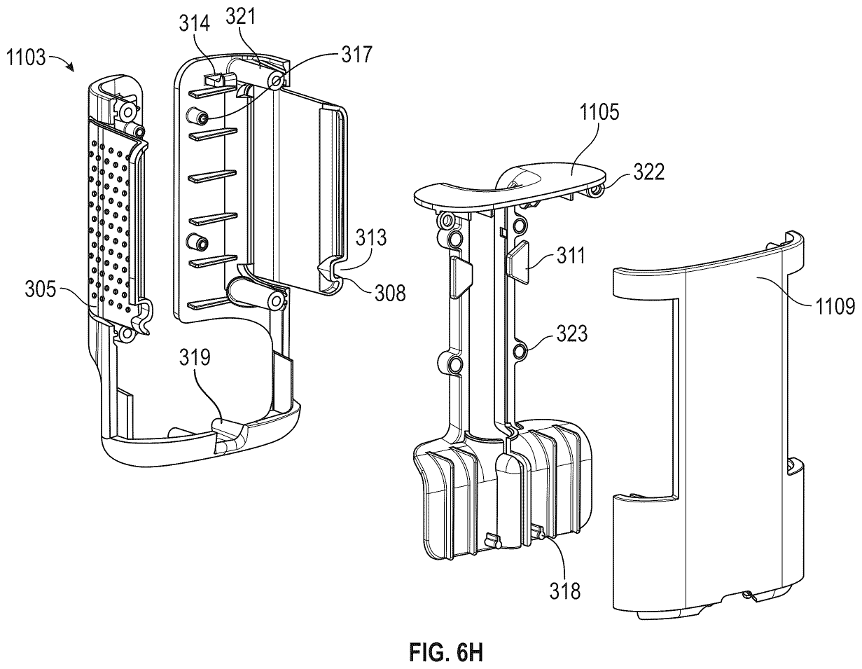

FIGS. 6A-6H illustrate views of example implementations of a transducer shroud 201. The example transducer shroud 201 may include similar structural and/or operational features described with reference to example transducer 101 of FIG. 1. FIGS. 6A-6C illustrate an example shroud system 202 including a transducer 213, a transducer shroud 201 mounted to a mounting plate 301 through engagement of a transducer wings 305 (e.g., a movable arm and/or the like) with mounting plate brackets 303 (e.g., a bracket arm and/or the like). The transducer shroud 201 may be configured to hold a transducer 213 coupled to a patient. In some examples, the shroud 201 can have a molded plastic housing including a thermoplastic grade material. In some examples, the molded plastic can be a blend of polycarbonate (PC) and/or acrylonitrile Butadiene Styrene (ABS). FIG. 6D illustrates an exploded view of the shroud system 202 of FIGS. 6A-6C, that includes a transducer 213 and transducer shroud 201. As shown, the shroud 201 may include one or more components for holding and/or coupling a transducer 213. In some examples, the transducer shroud 201 may include a back securing portion 304 configured to secure one or more parts of the transducer 213 and/or cannula tubing to prevent unintentional forward and/or backward movement of the transducer 213. In some examples, the back securing portion 304 may not include a front securing portion so as to allow easier access to components of the transducer 213.

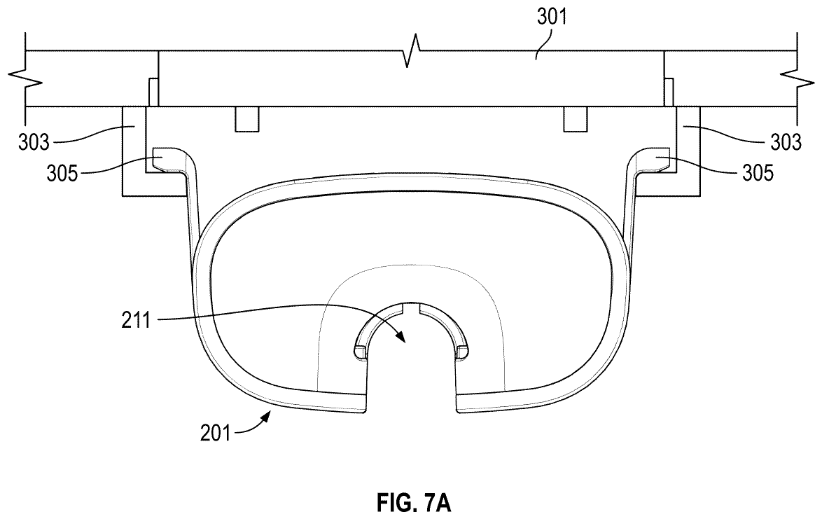

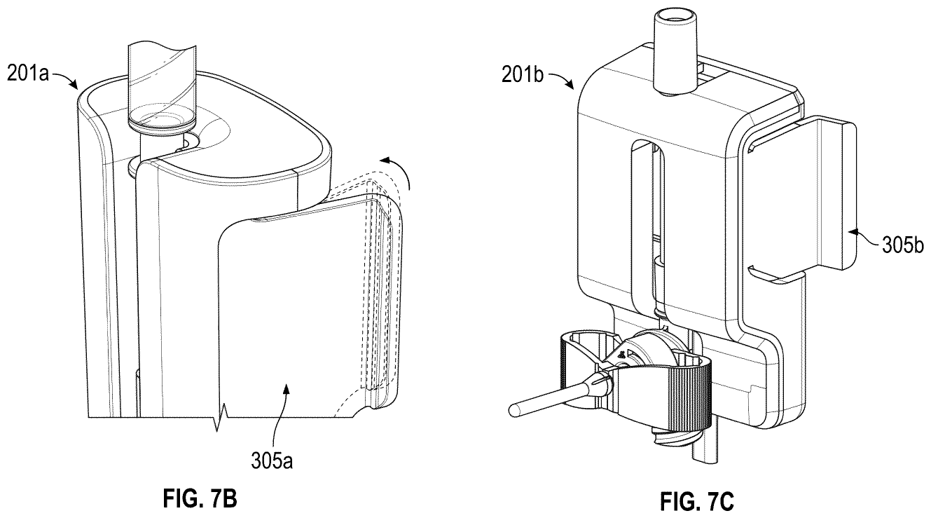

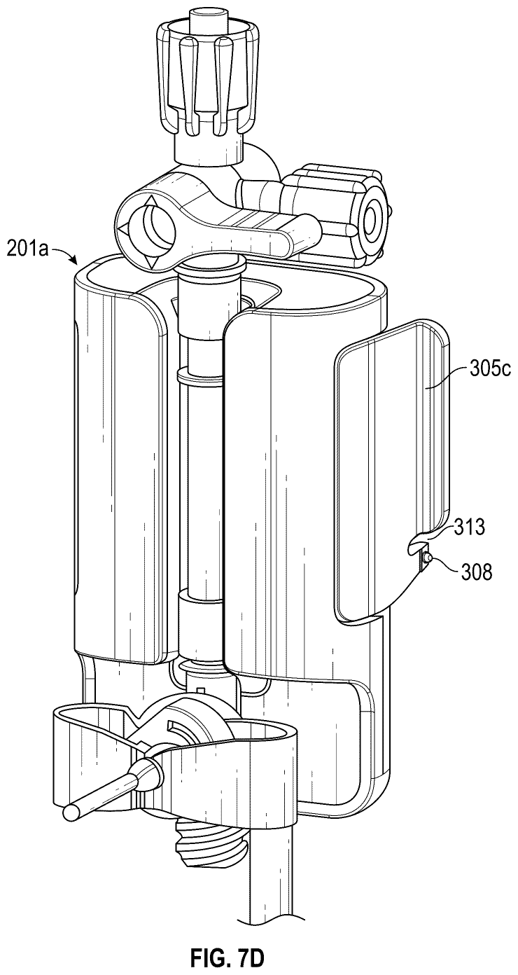

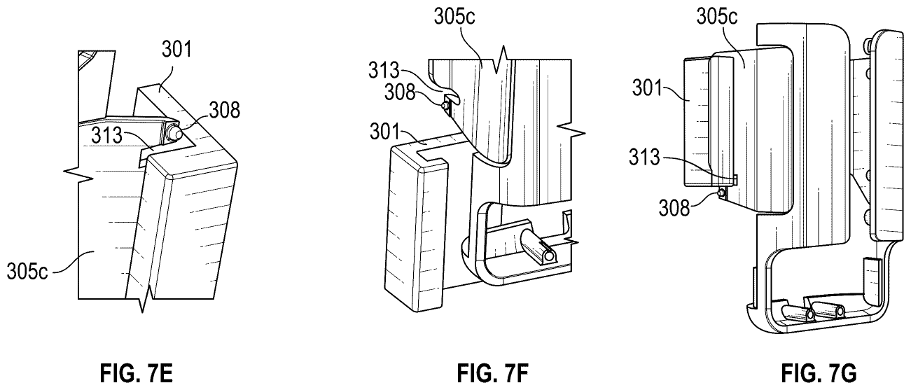

FIG. 6E illustrates an exploded view of the shroud 201 and a transducer 213. As illustrated, the shroud 201 may have a plurality of components, including but not limited to a back plate or back component 1109 configured to receive the transducer 213. The shroud 201 may have a middle component 1105 configured to secure the transducer 213 to within the shroud 201. The shroud 201 may include a cover plate or cover component 1103 configured to cover the top of the transducer 213. The cover component 1103 may include wings 305 such as described in FIGS. 7A-7C below.

The back component 1109 can include pins 309, a coupling component 310, a bottom lip 316, and support posts 320.

Pins 309 may be disposed in the interior portion of the back component 1109. The pins 309 can be positioned on the interior portion such that they align with corresponding receiving ends 321 of cover component 1103 and corresponding loops 322 of middle component 1105. For example, pins 309 may be disposed at or near the perimeter of the interior portion of the back component 1109. In some examples, pins 309 may be disposed at or near an edge of the inner portion. In some examples, pins 309 may be disposed at a top portion and/or bottom portion of the interior portion of back component 1109. Pins 309 may extend from the interior portion of back component 1109. In some aspects, the pins 309 may extend along an axis aligned with that of one or more receiving ends 321 of the cover component 1103 and/or one or more loops 322 of the middle component 1105. Pins 309 may be spaced from each other.

Pins 309 may be solid or hollow. Pins 309 may be cylindrical in shape and may have the same diameter as, or a slightly larger diameter than, an inner diameter of the receiving end 321 and/or of loops 322 to allow for a friction-fit connection. However, the cylindrical shape is not intended to be limiting. Pins 309 may embody any shape, such as any shape that can be received within receiving ends 321 of the cover component 1103 and/or loops 322 of the middle component 1105. In some aspects, pins 309 may be tapered for a tight fit when received by the receiving ends 321. In some aspects, the pins 309 may include threads and can be screwed into receiving ends 321 of the cover component 1103 and/or or secured with a nut. In some aspects, pins 309 can include a snap hook (e.g., snap-fit) mechanism, a latch, or interlocking tabs and/or grooves such as a ribbed or barbed shaft that grips the inner and/or outer sides of the receiving ends 321. In some aspects, at least a portion of the shafts of pins 309 may be magnetic to secure to the receiving ends 321 via a magnetic force.

Pins 309 can be configured to secure via a friction force and/or a snap-fit, latch, and/or the like, to receiving ends 321 of the cover component 1103. In some aspects, the pins 309 may be received through loops 322 of the middle component 1105. When secured, pins 309 can help prevent a shroud 201 from disassembly during use. Advantageously, pins 309 can be used in substitution and/or in conjunction with other means of adhering one or more components of the shroud 201 (e.g., such as applying a glue between one or more components to provide a friction fit, securing the back component 1109 to the middle component 1105 and/or the cover component 1103).

Coupling component 310 may be disposed in the interior of the back component 1109. The coupling component 310 may be positioned on the interior portion such that it aligns with, for example, a receiving end of the middle component 1105. For example, the coupling component 310 may be disposed at or near the perimeter of the interior portion of the back component 1109. In some examples, the coupling component 310 may be disposed at or near a top portion of the interior portion. In some examples, the coupling component 310 may be disposed at or near an edge (e.g., a top edge) of the top portion. The coupling component 310 may be disposed centrally within the top portion of the interior portion of back component 1109.

In some aspects, the coupling component 310 can include a snap-fit feature such as a snap hook or a mechanical fastener such as a latch. In some aspects, the coupling component 310 can include threads and be screwed into a receiving end of the middle component 1105 and/or secured via a nut. The coupling component 310 can include a fastener that secures the back component 1109 to the middle component 1105 via a twisting motion (e.g., twist-and-lock). In some aspects, the coupling component 310 can include interlocking tabs and/or grooves such as a ribs or barbs that grip the sides of a receiving end of middle component 1105. The coupling component 310 may include magnets to secure the back component 1109 to the middle component 1105 via a magnetic force.

The coupling component 310 can provide a latch to secure the back component 1109 to the middle component 1105 and/or the cover component 1103. Coupling component 310 can help ensure that the back component 1109 is aligned with the middle component 1105 and/or the cover component 1103 when the back component 1109 is secured to the middle component 1105 and/or the cover component 1103. Advantageously, coupling component 310 can be used in substitution and/or in conjunction with other means of adhering one or more components of the shroud 201 (e.g., such as applying a glue between one or more components to provide a friction fit, securing the back component 1109 to the middle component 1105 and/or the cover component 1103).

The bottom lip 316 may be disposed inward of the interior portion of the back component 1109. The bottom lip 316 may be disposed at or near a bottom portion of the interior portion of back component 1109. In some aspects, the bottom lip 316 may extend along at least a portion of the bottom portion of the interior portion of back component 1109. For example, the bottom lip 316 may extend along at least a portion of a bottom edge of the bottom portion. In some examples, the bottom lip 316 may extend along the entirety of the bottom edge of the bottom portion of the interior portion. In some aspects, the bottom lip 316 may include two or more ridges, each of which extend along at least a portion of the bottom portion of the interior portion. For example, ridges(s) of the bottom lip 316 may extend from (or from inward of) a side edge of the bottom portion to a middle point (or to inward of a middle point) of the bottom portion. In some aspects, ridge(s) of the bottom lip 316 may be spaced apart from each other. In some aspects, ridge(s) of the bottom lip 316 may be connected to one another.

The bottom lip 316 can provide one or more ridges that extend beyond at least a portion of the back component 1109. For example, the ridge(s) may extend beyond at least a portion of the bottom portion of back component 1109. In some examples, the ridge(s) may extend beyond at least a portion of the bottom edge of the bottom portion. The ridge(s) may extend in a direction opposite of a top portion of the back component 1109. The bottom lip 316 may include rigid or semi-rigid material such as on the ridge(s) of the bottom lip such that the ridge(s) may be prohibited from flexing (or may flex a certain amount) upon contact with the middle component 1105 and/or the cover component 1103. In some aspects, the bottom lip 316 may include one or more coupling components such as on the ridge(s), including any of the coupling components described herein.

The bottom lip 316 can be configured to secure via a friction force and/or a snap, latch, and/or the like, to a bottom groove (e.g., bottom groove 315) of the cover component 1103 to prevent the cover component 1103 and back component 1109 from disassembly during use. The bottom lip 316 and bottom groove 315 and can help ensure that the back component 1109 is aligned with the cover component 1103 when the back component 1109 and cover component 1103 are secured to one another. Advantageously, bottom lip 316 can be used in substitution and/or in conjunction with other means of adhering one or more components of the shroud 201 (e.g., such as applying a glue between one or more components to provide a friction fit, securing the back component 1109 to the middle component 1105 and/or the cover component 1103).

Support posts 320 may be disposed in the interior portion of the back component 1109. Posts 320 may be disposed at a middle portion of the interior portion. In some aspects, posts 320 may be positioned on the interior portion of the back component 1109 such that the posts 320 contact the transducer 213 when the transducer shroud 201 holds the transducer 213. Posts 320 may extend from the interior portion of back component 1109. In some aspects, posts 320 may be spaced from each other. For example, posts 320 may be spaced equidistant from each other. Support posts 320 may be solid or hollow. Posts 320 may be cylindrical in shape and have the same diameter or a different diameter than those of pins 309. For example, posts 320 may be larger in diameter than are pins 309. However, the cylindrical shape is not intended to be limiting. Posts 320 may embody any shape, such as any shape that can contact and support the transducer 213 while held by the shroud 201. The support posts 320 can be configured to contact and support the transducer 213 and ensure that the transducer 213 is secured and remains in a fixed position while in use (e.g., while the shroud 201 holds the transducer 213) while preventing unintended separation.

The cover component 1103 can include a bottom groove 315, one or more receiving ends 319, and one or more receiving ends 321. In some embodiments, the cover component 1103 can include one or more coupling components such as coupling component 314 shown and/or described in FIGS. 6G-6H. In some embodiments, the cover component 1103 can include one or more securing posts such as securing posts 317 shown and/or described in FIG. 6H.

The bottom groove 315 may be disposed at or near a bottom portion of the cover component 1103. The bottom groove 315 may extend along at least a portion of the bottom portion of the cover component 1103. For example, the bottom groove 315 may extend along at least a portion of a bottom edge of the bottom portion. In some examples, the bottom groove 315 may extend along the entirety of the bottom edge of the bottom portion of the interior portion.

The bottom groove 315 can provide one or more ridges that extend from the bottom portion of the interior portion of the cover component 1103. In some aspects, the ridge(s) may extend from the bottom edge of the bottom portion toward a top portion of the inner portion of the cover component 1103. The ridge(s) may be disposed inward of the inner portion of the cover component 1103. In some aspects, the ridge(s) may extend along at least a portion of the bottom portion of the cover component 1103. For example, the ridge(s) may extend along at least a portion of the bottom edge of the bottom portion. In some examples, the ridge(s) may extend along the entirety of the bottom edge of the bottom portion of the interior portion of cover component 1103. In some aspects, the ridges may be spaced apart from each other. In some aspects, the ridges may be connected to each other. The bottom groove 315 may include rigid or semi-rigid material such as on the ridge(s) of the bottom groove 315 such that the ridge(s) may be prohibited from flexing (or may flex a certain amount) upon contact with the middle component 1105 and/or the back component 1109. In some aspects, the bottom groove 315 may include one or more coupling components such as on the ridge(s), including any of the coupling components described herein.

The bottom groove 315 may fit and/or secure to bottom lip 316 of the back component 1109. The bottom groove 315 and bottom lip 316 can help ensure that the cover component 1103 is aligned with the back component 1109 when the cover component 1103 and back component 1109 are secured to one another. Advantageously, bottom groove 315 can be used in substitution and/or in conjunction with other means of adhering one or more components of the shroud 201 (e.g., such as applying a glue between one or more components to provide a friction fit, securing the cover component 1103 to the middle component 1105 and/or the back component 1109).

Receiving ends 319 may be positioned at or near the bottom edge of the bottom portion of the cover component 1103. For example, the receiving ends 319 may be disposed within bottom groove 315. Receiving ends 319 may be disposed inward of the ridge(s) of bottom groove 315 and may connect to the ridge(s). Receiving ends 319 may be spaced from each other. In some aspects, the receiving ends 319 may extend along an axis different from the axis along which the bottom edge of the bottom portion extends. For example, the receiving ends 319 may extend along an axis that is perpendicular to the axis along which the bottom edge of the bottom portion extends. The receiving ends 319 may extend along an axis aligned with that of one or more pins, such as pins 318 of the middle component 1105 shown and/or described in FIG. 6H. Receiving ends 319 may be spaced from each other.

Receiving ends 319 may be hollow. In some aspects, receiving ends 319 may be cylindrical in shape. Receiving ends 319 can have a middle-facing surface (e.g., facing toward the middle component when the cover component is secured to the middle component). Receiving ends 319 can include an outer sidewall extending between the ridge of bottom groove 315 and the middle-facing surface. The outer sidewall can include an outer edge having a constant or substantially constant outer diameter. In some aspects, the receiving ends 319 may be tapered such that the outer diameter at a first portion of the receiving ends 319 is different than the outer diameter at a second portion of the receiving ends 319. Receiving ends 319 can include an inner sidewall extending between the ridge of bottom groove 315 and the middle-facing surface. The inner sidewall can include an inner edge having a constant or substantially constant inner diameter. In some aspects, the receiving ends 319 may be tapered such that the inner diameter at a first portion of the receiving ends 319 is different than the inner diameter at a second portion of the receiving ends 319. The outer diameter can be greater than the inner diameter. The middle-facing surface can extend between the outer and inner edges. Receiving ends 319 may have an inner diameter that is the same or less than a diameter of a corresponding coupling component (e.g., pins 318) to allow for a friction-fit connection. However, the cylindrical shape is not intended to be limiting. Receiving ends 319 may embody any shape, such as any shape that can receive coupling or alignment components (e.g., pins 318) of middle component 1105 and/or back component 1109. In some aspects, receiving ends 319 may be tapered for a tight fit when receiving a coupling component. In some aspects, receiving ends 319 can include threads such that a coupling component can screw into the ends 319, or any other coupling mechanism described herein. In some aspects, receiving ends 319 can include a groove or slot, such as an alignment slot, which can receive a protrusion including a ridge or rail of a coupling component of middle component 1105 (such as pins 318 shown and/or described in FIG. 6H) and/or back component 1109. For example, receiving ends 319 may fit or secure to pins 318.

Receiving ends 319 can be configured to secure via a friction force and/or a snap-fit, latch, and/or the like, to a coupling component of the middle component 1105 and/or the back component 1109 to prevent a shroud 201 from disassembly during use. Receiving ends 319 and pins such as pins 318 can help ensure that the cover component 1103 is aligned with the middle component 1105 and/or the back component 1109 when the cover component 1103 is secured to the middle component 1105 and/or the back component 1109. Advantageously, receiving ends 319 can be used in substitution and/or in conjunction with other means of adhering one or more components of the shroud 201 (e.g., such as applying a glue between one or more components to provide a friction fit, securing the cover component 1103 to the middle component 1105 and/or the back component 1109).

Receiving ends 321 may be disposed in the interior portion of the cover component 1103. The receiving ends 321 can be positioned on the interior portion of the cover component 1103 such that they align with corresponding pins 309 of back component 1109 and corresponding loops 322 of middle component 1105. For example, receiving ends 321 may be disposed at a top portion and/or bottom portion of the interior portion of cover component 1103. In some examples, receiving ends 321 may be disposed at or near the perimeter of the interior portion of the cover component 1103. In some examples, receiving ends 321 may be disposed at or near an edge of the inner portion, such as a top edge or side edge of the cover component 1103. Receiving ends 321 may extend from the interior portion of cover component 1103. For example, receiving ends 321 may extend inward from the inner portion of the cover component 1103 in the direction of the back component 1109. In some aspects, the receiving ends 321 may extend along an axis aligned with that of one or more pins 309 of the back component 1109 and/or one or more loops 322 of the middle component 1105. Receiving ends 321 may be spaced from each other.

Receiving ends 321 may be hollow. In some aspects, receiving ends 321 may be cylindrical in shape. Receiving ends 321 can have a middle-facing surface (e.g., facing toward the middle component when the cover component is secured to the middle component). Receiving ends 321 can include an outer sidewall extending between a surface of the interior portion of the cover component 1103 and the middle-facing surface. The outer sidewall can include an outer edge having a constant or substantially constant outer diameter. In some aspects, the receiving ends 321 may be tapered such that the outer diameter at a first portion of the receiving ends 321 is different than the outer diameter at a second portion of the receiving ends 321. Receiving ends 321 can include an inner sidewall extending between the surface of the interior portion of the cover component 1103 and the middle-facing surface. The inner sidewall can include an inner edge having a constant or substantially constant inner diameter. In some aspects, the receiving ends 321 may be tapered such that the inner diameter at a first portion of the receiving ends 321 is different than the inner diameter at a second portion of the receiving ends 321. The outer diameter can be greater than the inner diameter. The middle-facing surface can extend between the outer and inner edges. Receiving ends 321 may have an inner diameter that is the same or less than a diameter of a corresponding coupling component (e.g., pins 309) to allow for a friction-fit connection. However, the cylindrical shape is not intended to be limiting. Receiving ends 321 may embody any shape, such as any shape that can receive coupling or alignment components (e.g., pins 309) of middle component 1105 and/or back component 1109. In some aspects, receiving ends 321 may be tapered for a tight fit when receiving a coupling component. In some aspects, receiving ends 321 can include threads such that a coupling component can screw into the ends 321, or any other coupling mechanism described herein. Receiving ends 321 may fit or secure to pins 309.

Receiving ends 321 can be configured to secure via a friction force and/or a snap-fit, latch, and/or the like, to a coupling component of the middle component 1105 and/or the back component 1109 to prevent a shroud 201 from disassembly during use. Receiving ends 321 and pins such as pins 309 can help ensure that the cover component 1103 is aligned with the middle component 1105 and/or the back component 1109 when the cover component 1103 is secured to the middle component 1105 and/or the back component 1109. Advantageously, receiving ends 321 can be used in substitution and/or in conjunction with other means of adhering one or more components of the shroud 201 (e.g., such as applying a glue between one or more components to provide a friction fit, securing the cover component 1103 to the middle component 1105 and/or the back component 1109).

The middle component 1105 can include an alignment component 311, loops 322, and receiving ends 323. In some embodiments, the middle component 1105 can include pins such as pins 318 shown and/or described in FIG. 6H.

The shroud 201 can be configured with one or more features to ensure that the shroud 201 is secured and aligned with a transducer 213. For example, the middle component 1105 may include an alignment component such as alignment lip 311. The alignment lips 311 may be positioned on the middle portion such that the alignment lips 311 aligns with an alignment slot such as alignment slots 312 of the transducer. For example, the alignment lips 311 may be disposed at a middle portion of the middle component 1105. In some examples, the alignment lips 311 may be disposed at or near an edge of the middle portion of middle component 1105. The alignment lips 311 may extend along at least a portion of the middle portion, such as a portion of an edge of the middle portion. In some examples, the alignment lips 311 may extend away from the middle portion of middle component 1105, such as outward laterally from an edge of the middle component 1105. The alignment lip 311 may fit and/or secure to the alignment slot 312 of the transducer 213. For example, the alignment lip 311 may be polygonal in shape, such as trapezoidal. However, this is not intended to be limiting. The alignment lip 311 may embody any shape that can be received by alignment slot 312. The alignment lip 311 and the slot 312 can ensure that the transducer 213 and the shroud 201 are secured and remain in a fixed position while in use.

The loops 322 may be positioned on the middle component 1105 such that they align with corresponding pins 309 of the back component 1109. For example, loops 322 may be disposed at an upper portion and/or bottom portion of the middle component 1105. In some examples, the loops 322 may be disposed at or near a perimeter of the middle component 1105. In some examples, the loops 322 may be disposed at or near and edge of the middle component 1105, such as a top edge or side edge of the middle component 1105. At least a portion of the loops 322 may extend away from the upper portion (and/or lower portion) of the middle component 1105, such as extending laterally from an edge of the middle component 1105. Loops 322 may be spaced from each other.

Loops 322 may be annular, ring-like, or cylindrical in shape. Loops 322 can have a cover-facing surface (e.g., facing toward the cover component when the middle component is secured to the cover component) and a back-facing surface (e.g., facing toward the back component when the middle component is secured to the back component). Loops 322 can include an outer sidewall extending between the cover-facing surface and the back-facing surface. The outer sidewall can include an outer edge having a constant or substantially constant inner diameter. In some aspects, the receiving ends 321 may be tapered such that the outer diameter at a first portion of the loops 322 is different than the outer diameter at a second portion of the loops 322. Loops 322 can include an inner sidewall extending between the cover-facing surface and the back-facing surface. The inner sidewall can include an inner edge having a constant or substantially constant inner diameter. In some aspects, the loops 322 may be tapered such that the inner diameter at a first portion of the loops 322 is different than the inner diameter at a second portion of the loops 322. The outer diameter can be greater than the inner diameter. The cover- and back-facing surfaces can extend between the outer and inner edges. The loops 322 may have an inner diameter that is the same as, or substantially the same as, a diameter of corresponding pins 309 to allow for a friction fit connection and/or to allow pins 309 to pass through loops 322. In some embodiments, the loops 322 may have an inner diameter that is larger or smaller (e.g., slightly larger or smaller) than a diameter of pins 309. In the example of FIGS. 6E-6H, the loops 322 are annular rings. However, this is not intended to be limiting. Loops 322 may embody any shape, such as any shape that can receive coupling or alignment components (e.g., pins 309) of back component 1109 and/or cover component 1103.

Loops 322 can be configured to ensure that the back component 1109 is secured and aligned with the middle component 1105, such as when the back component 1109 is secured to the middle component 1105 and/or to the cover component 1103. The loops 322 can be configured to ensure that the pins 309 of the back component 1109 are aligned with the receiving ends 321 of the cover component 1103, such as when the back component 1109 is secured to the cover component 1103. Advantageously, loops 322 can be configured to reduce lift and angular moment of rotation of the back component 1109 when the back component 1109 is secured to the middle component 1105 and and/or secured to the cover component 1103. Advantageously, loops 322 can be used in substitution and/or in conjunction with other means of adhering one or more components of the shroud 201 (e.g., such as applying a glue between one or more components to provide a friction fit, securing the back component 1109 to the middle component 1105 and/or the cover component 1103).

Receiving ends 323 can be positioned on the middle component 1105 such that they align with a coupling or alignment component, such as securing posts 317 of the cover component 1103 shown and/or described in FIG. 6H. For example, the receiving ends 323 may be disposed at a top portion and/or middle portion of the middle component 1105. In some examples, receiving ends 323 may be disposed at or near a perimeter of the middle component 1105. In some examples, the receiving ends 323 may be disposed at or near an edge of the middle component 1105, such as a top edge or side edge of middle component 1105. At least a portion of the receiving ends 323 may extend away from the upper portion and/or middle portion of middle component 1105, such as extending laterally from an edge of the middle component 1105. In some aspects, the receiving ends 323 may extend along an axis aligned with that of one or more coupling or alignment components (e.g., securing posts 317) of the cover component 1103 and/or of the back component 1109. Receiving ends 323 may be spaced from each other.

Receiving ends 323 may be hollow. In some aspects, receiving ends 323 may be cylindrical in shape. Receiving ends 323 can have a cover-facing surface (e.g., facing toward the cover component when the middle component is secured to the cover component) and a back-facing surface (e.g., facing toward the back component when the middle component is secured to the cover component). Receiving ends 323 can include an outer sidewall extending between the cover-facing surface and the back-facing surface. The outer sidewall can include an outer edge having a constant or substantially constant outer diameter. In some aspects, the receiving ends 323 may be tapered such that the outer diameter at a first portion of the receiving ends 323 is different than the outer diameter at a second portion of the receiving ends 323. Receiving ends 323 can include an inner sidewall extending between the cover-facing surface and the back-facing surface. The inner sidewall can include an inner edge having a constant or substantially constant inner diameter. In some aspects, the receiving ends 323 may be tapered such that the inner diameter at a first portion of the receiving ends 323 is different than the inner diameter at a second portion of the receiving ends 323. The outer diameter can be greater than the inner diameter. The cover- and back-facing surfaces can extend between the outer and inner edges. Receiving ends 323 may have an inner diameter that is the same or less than an outer diameter of a corresponding coupling component or alignment component (e.g., securing posts 317) to allow for a friction-fit connection. However, the cylindrical shape is not intended to be limiting. Receiving ends 323 may embody any shape, such as any shape that can receive coupling or alignment components (e.g., securing posts 317) of cover component 1103 and/or back component 1109. In some aspects, receiving ends 323 may be tapered for a tight fit when receiving a coupling or alignment component. In some aspects, receiving ends 323 can include threads such that a coupling component can screw into the ends 323, or any other coupling mechanism described herein. Receiving ends 323 may fit or secure to securing posts, such as securing posts 317.

Receiving ends 323 can be configured to secure via a friction force and/or a snap-fit, latch, and/or the like, to a coupling or alignment component of the front component 1103 and/or the back component 1109 to prevent a shroud 201 from disassembly during use. Receiving ends 323 and securing posts such as securing posts 317 can help ensure that the cover component 1103 is aligned with the middle component 1105 and/or the back component 1109 when the cover component 1103 is secured to the middle component 1105 and/or the back component 1109. Advantageously, receiving ends 323 can be used in substitution and/or in conjunction with other means of adhering one or more components of the shroud 201 (e.g., such as applying a glue between one or more components to provide a friction fit, securing the cover component 1103 to the middle component 1105 and/or the back component 1109).

In some implementations, once secured, the locking features of shroud 201 may prevent separation of shroud components or opening of the shroud without forceful breaking of the shroud 201. In some implementations, one or more of the features mentioned herein (e.g., pins 309, coupling component 310, alignment lip 311, slot 312, coupling component 314, bottom groove 315, bottom lip 316, securing posts 317, pins 318, receiving ends 319, support posts 320, receiving ends 321, loops 322, receiving ends 323, and/or the like) may be used to secure a shroud 201 in substitution for applying an adhesive substance (e.g., glue and/or the like).

FIG. 6F illustrates an exploded view of an additional implementation of the shroud 201. As illustrated, the shroud 201 may have a plurality of components, including but not limited to a back plate or back component 1109 configured to receive the transducer 213. The shroud 201 may have a middle component 1105 configured to secure the transducer 213 to within the shroud 201. The shroud 201 may include a cover plate or cover component 1103a configured to cover the top of the transducer 213. The shroud 201 of FIG. 6F may include one or more features to ensure that a shroud 201 is secured and aligned with a transducer 213 as described with reference to FIG. 6E. For example, the cover component 1103 may include wings 305c such as described in FIGS. 7A-7C below. The cover component 1103a may include a securing device 308 and/or a slot 313 further described in FIGS. 7D-7G.

The back component 1109a can include one or more features such as described with reference to FIG. 6E. For example, back component 1109a can include pins 309, a coupling component 310, a bottom lip 316, and support posts 320.

Pins 309 may be disposed in the interior portion of the back component 1109a. The pins 309 can be positioned on the interior portion such that they align with corresponding receiving ends 321 of cover component 1103a and corresponding loops 322 of middle component 1105a. For example, pins 309 may be disposed at or near the perimeter of the interior portion of the back component 1109a. In some examples, pins 309 may be disposed at or near an edge of the inner portion. In some examples, pins 309 may be disposed at a top portion and/or bottom portion of the interior portion of back component 1109a. Pins 309 may extend from the interior portion of back component 1109a. In some aspects, the pins 309 may extend along an axis aligned with that of one or more receiving ends 321 of the cover component 1103a and/or one or more loops 322 of the middle component 1105. Pins 309 may be spaced from each other.

Pins 309 may be solid or hollow. Pins 309 may be cylindrical in shape and may have the same diameter as, or a slightly larger diameter than, an inner diameter of the receiving end 321 and/or of loops 322 to allow for a friction-fit connection. However, the cylindrical shape is not intended to be limiting. Pins 309 may embody any shape, such as any shape that can be received within receiving ends 321 of the cover component 1103 and/or loops 322 of the middle component 1105. In some aspects, pins 309 may be tapered for a tight fit when received by the receiving ends 321. In some aspects, the pins 309 may include threads and can be screwed into receiving ends 321 of the cover component 1103a and/or or secured with a nut. In some aspects, pins 309 can include a snap hook (e.g., snap-fit) mechanism, a latch, or interlocking tabs and/or grooves such as a ribbed or barbed shaft that grips the inner and/or outer sides of the receiving ends 321. In some aspects, at least a portion of the shafts of pins 309 may be magnetic to secure to the receiving ends 321 via a magnetic force.

Pins 309 can be configured to secure via a friction force and/or a snap-fit, latch, and/or the like, to receiving ends 321 of the cover component 1103a. In some aspects, the pins 309 may be received through loops 322 of the middle component 1105a. When secured, pins 309 can help prevent a shroud 201 from disassembly during use. Advantageously, pins 309 can be used in substitution and/or in conjunction with other means of adhering one or more components of the shroud 201 (e.g., such as applying a glue between one or more components to provide a friction fit, securing the back component 1109a to the middle component 1105a and/or the cover component 1103a).

Coupling component 310 may be disposed in the interior of the back component 1109a. The coupling component 310 may be positioned on the interior portion such that it aligns with, for example, a receiving end of the middle component 1105a. For example, the coupling component 310 may be disposed at or near the perimeter of the interior portion of the back component 1109a. In some examples, the coupling component 310 may be disposed at or near a top portion of the interior portion. In some examples, the coupling component 310 may be disposed at or near an edge (e.g., a top edge) of the top portion. The coupling component 310 may be disposed centrally within the top portion of the interior portion of back component 1109a.

In some aspects, the coupling component 310 can include a snap-fit feature such as a snap hook or a mechanical fastener such as a latch. In some aspects, the coupling component 310 can include threads and be screwed into a receiving end of the middle component 1105a and/or secured via a nut. The coupling component 310 can include a fastener that secures the back component 1109a to the middle component 1105a via a twisting motion (e.g., twist-and-lock). In some aspects, the coupling component 310 can include interlocking tabs and/or grooves such as a ribs or barbs that grip the sides of a receiving end of middle component 1105a. The coupling component 310 may include magnets to secure the back component 1109a to the middle component 1105a via a magnetic force.

The coupling component 310 can provide a latch to secure the back component 1109a to the middle component 1105a and/or the cover component 1103. Coupling component 310 can help ensure that the back component 1109a is aligned with the middle component 1105a and/or the cover component 1103a when the back component 1109a is secured to the middle component 1105a and/or the cover component 1103a. Advantageously, coupling component 310 can be used in substitution and/or in conjunction with other means of adhering one or more components of the shroud 201 (e.g., such as applying a glue between one or more components to provide a friction fit, securing the back component 1109a to the middle component 1105a and/or the cover component 1103a).

The bottom lip 316 may be disposed inward of the interior portion of the back component 1109a. The bottom lip 316 may be disposed at or near a bottom portion of the interior portion of back component 1109a. In some aspects, the bottom lip 316 may extend along at least a portion of the bottom portion of the interior portion of back component 1109a. For example, the bottom lip 316 may extend along at least a portion of a bottom edge of the bottom portion. In some examples, the bottom lip 316 may extend along the entirety of the bottom edge of the bottom portion of the interior portion. In some aspects, the bottom lip 316 may include two or more ridges, each of which extend along at least a portion of the bottom portion of the interior portion. For example, ridges(s) of the bottom lip 316 may extend from (or from inward of) a side edge of the bottom portion to a middle point (or to inward of a middle point) of the bottom portion. In some aspects, ridge(s) of the bottom lip 316 may be spaced apart from each other. In some aspects, ridge(s) of the bottom lip 316 may be connected to one another.

The bottom lip 316 can provide one or more ridges that extend beyond at least a portion of the back component 1109a. For example, the ridge(s) may extend beyond at least a portion of the bottom portion of back component 1109a. In some examples, the ridge(s) may extend beyond at least a portion of the bottom edge of the bottom portion. The ridge(s) may extend in a direction opposite of a top portion of the back component 1109a. The bottom lip 316 may include rigid or semi-rigid material such as on the ridge(s) of the bottom lip such that the ridge(s) may be prohibited from flexing (or may flex a certain amount) upon contact with the middle component 1105a and/or the cover component 1103a. In some aspects, the bottom lip 316 may include one or more coupling components such as on the ridge(s), including any of the coupling components described herein.

The bottom lip 316 can be configured to secure via a friction force and/or a snap, latch, and/or the like, to a bottom groove (e.g., bottom groove 315) of the cover component 1103a to prevent the cover component 1103a and back component 1109a from disassembly during use. The bottom lip 316 and bottom groove 315 and can help ensure that the back component 1109a is aligned with the cover component 1103a when the back component 1109a and cover component 1103a are secured to one another. Advantageously, bottom lip 316 can be used in substitution and/or in conjunction with other means of adhering one or more components of the shroud 201 (e.g., such as applying a glue between one or more components to provide a friction fit, securing the back component 1109a to the middle component 1105a and/or the cover component 1103a).

Support posts 320 may be disposed in the interior portion of the back component 1109a. Posts 320 may be disposed at a middle portion of the interior portion. In some aspects, posts 320 may be positioned on the interior portion of the back component 1109a such that the posts 320 contact the transducer 213 when the transducer shroud 201 holds the transducer 213. Posts 320 may extend from the interior portion of back component 1109a. In some aspects, posts 320 may be spaced from each other. For example, posts 320 may be spaced equidistant from each other. Support posts 320 may be solid or hollow. Posts 320 may be cylindrical in shape and have the same diameter or a different diameter than those of pins 309. For example, posts 320 may be larger in diameter than are pins 309. However, the cylindrical shape is not intended to be limiting. Posts 320 may embody any shape, such as any shape that can contact and support the transducer 213 while held by the shroud 201. The support posts 320 can be configured to contact and support the transducer 213 and ensure that the transducer 213 is secured and remains in a fixed position while in use (e.g., while the shroud 201 holds the transducer 213) while preventing unintended separation.

The cover component 1103a can include one or more features such as described with reference to FIG. 6E. For example, cover component 1103a can include a bottom groove 315, one or more receiving ends 319, and one or more receiving ends 321. In some embodiments, the cover component 1103a can include one or more coupling components such as coupling component 314 shown and/or described in FIGS. 6G-6H. In some embodiments, the cover component 1103a can include one or more securing posts such as securing posts 317 shown and/or described in FIG. 6H.

The bottom groove 315 may be disposed at or near a bottom portion of the cover component 1103a. The bottom groove 315 may extend along at least a portion of the bottom portion of the cover component 110a3. For example, the bottom groove 315 may extend along at least a portion of a bottom edge of the bottom portion. In some examples, the bottom groove 315 may extend along the entirety of the bottom edge of the bottom portion of the interior portion.

The bottom groove 315 can provide one or more ridges that extend from the bottom portion of the interior portion of the cover component 1103a. In some aspects, the ridge(s) may extend from the bottom edge of the bottom portion toward a top portion of the inner portion of the cover component 1103a. The ridge(s) may be disposed inward of the inner portion of the cover component 1103a. In some aspects, the ridge(s) may extend along at least a portion of the bottom portion of the cover component 1103. For example, the ridge(s) may extend along at least a portion of the bottom edge of the bottom portion. In some examples, the ridge(s) may extend along the entirety of the bottom edge of the bottom portion of the interior portion of cover component 1103a. In some aspects, the ridges may be spaced apart from each other. In some aspects, the ridges may be connected to each other. The bottom groove 315 may include rigid or semi-rigid material such as on the ridge(s) of the bottom groove 315 such that the ridge(s) may be prohibited from flexing (or may flex a certain amount) upon contact with the middle component 1105a and/or the back component 1109a. In some aspects, the bottom groove 315 may include one or more coupling components such as on the ridge(s), including any of the coupling components described herein.

The bottom groove 315 may fit and/or secure to bottom lip 316 of the back component 1109a. The bottom groove 315 and bottom lip 316 can help ensure that the cover component 1103a is aligned with the back component 1109a when the cover component 1103a and back component 1109a are secured to one another. Advantageously, bottom groove 315 can be used in substitution and/or in conjunction with other means of adhering one or more components of the shroud 201 (e.g., such as applying a glue between one or more components to provide a friction fit, securing the cover component 1103a to the middle component 1105a and/or the back component 1109a).

Receiving ends 319 may be positioned at or near the bottom edge of the bottom portion of the cover component 1103a. For example, the receiving ends 319 may be disposed within bottom groove 315. Receiving ends 319 may be disposed inward of the ridge(s) of bottom groove 315 and may connect to the ridge(s). Receiving ends 319 may be spaced from each other. In some aspects, the receiving ends 319 may extend along an axis different from the axis along which the bottom edge of the bottom portion extends. For example, the receiving ends 319 may extend along an axis that is perpendicular to the axis along which the bottom edge of the bottom portion extends. The receiving ends 319 may extend along an axis aligned with that of one or more pins, such as pins 318 of the middle component 1105 shown and/or described in FIG. 6H. Receiving ends 319 may be spaced from each other.

Receiving ends 319 may be hollow. In some aspects, receiving ends 319 may be cylindrical in shape. Receiving ends 319 can have a middle-facing surface (e.g., facing toward the middle component when the cover component is secured to the middle component). Receiving ends 319 can include an outer sidewall extending between the ridge of bottom groove 315 and the middle-facing surface. The outer sidewall can include an outer edge having a constant or substantially constant outer diameter. In some aspects, the receiving ends 319 may be tapered such that the outer diameter at a first portion of the receiving ends 319 is different than the outer diameter at a second portion of the receiving ends 319. Receiving ends 319 can include an inner sidewall extending between the ridge of bottom groove 315 and the middle-facing surface. The inner sidewall can include an inner edge having a constant or substantially constant inner diameter. In some aspects, the receiving ends 319 may be tapered such that the inner diameter at a first portion of the receiving ends 319 is different than the inner diameter at a second portion of the receiving ends 319. The outer diameter can be greater than the inner diameter. The middle-facing surface can extend between the outer and inner edges. Receiving ends 319 may have an inner diameter that is the same or less than a diameter of a corresponding coupling component (e.g., pins 318) to allow for a friction-fit connection. However, the cylindrical shape is not intended to be limiting. Receiving ends 319 may embody any shape, such as any shape that can receive coupling or alignment components (e.g., pins 318) of middle component 1105a and/or back component 1109a. In some aspects, receiving ends 319 may be tapered for a tight fit when receiving a coupling component. In some aspects, receiving ends 319 can include threads such that a coupling component can screw into the ends 319, or any other coupling mechanism described herein. In some aspects, receiving ends 319 can include a groove or slot, such as an alignment slot, which can receive a protrusion including a ridge or rail of a coupling component of middle component 1105a (such as pins 318 shown and/or described in FIG. 6H) and/or back component 1109a. For example, receiving ends 319 may fit or secure to pins 318.

Receiving ends 319 can be configured to secure via a friction force and/or a snap-fit, latch, and/or the like, to a coupling component of the middle component 1105a and/or the back component 1109a to prevent a shroud 201 from disassembly during use. Receiving ends 319 and pins such as pins 318 can help ensure that the cover component 1103a is aligned with the middle component 1105a and/or the back component 1109a when the cover component 1103a is secured to the middle component 1105a and/or the back component 1109a. Advantageously, receiving ends 319 can be used in substitution and/or in conjunction with other means of adhering one or more components of the shroud 201 (e.g., such as applying a glue between one or more components to provide a friction fit, securing the cover component 1103a to the middle component 1105a and/or the back component 1109a).

Receiving ends 321 may be disposed in the interior portion of the cover component 1103. The receiving ends 321 can be positioned on the interior portion of the cover component 1103a such that they align with corresponding pins 309 of back component 1109a and corresponding loops 322 of middle component 1105a. For example, receiving ends 321 may be disposed at a top portion and/or bottom portion of the interior portion of cover component 1103a. In some examples, receiving ends 321 may be disposed at or near the perimeter of the interior portion of the cover component 1103a. In some examples, receiving ends 321 may be disposed at or near an edge of the inner portion, such as a top edge or side edge of the cover component 1103a. Receiving ends 321 may extend from the interior portion of cover component 1103a. For example, receiving ends 321 may extend inward from the inner portion of the cover component 1103a in the direction of the back component 1109a. In some aspects, the receiving ends 321 may extend along an axis aligned with that of one or more pins 309 of the back component 1109a and/or one or more loops 322 of the middle component 1105a. Receiving ends 321 may be spaced from each other.

Receiving ends 321 may be hollow. In some aspects, receiving ends 321 may be cylindrical in shape. Receiving ends 321 can have a middle-facing surface (e.g., facing toward the middle component when the cover component is secured to the middle component). Receiving ends 321 can include an outer sidewall extending between a surface of the interior portion of the cover component 1103a and the middle-facing surface. The outer sidewall can include an outer edge having a constant or substantially constant outer diameter. In some aspects, the receiving ends 321 may be tapered such that the outer diameter at a first portion of the receiving ends 321 is different than the outer diameter at a second portion of the receiving ends 321. Receiving ends 321 can include an inner sidewall extending between the surface of the interior portion of the cover component 1103a and the middle-facing surface. The inner sidewall can include an inner edge having a constant or substantially constant inner diameter. In some aspects, the receiving ends 321 may be tapered such that the inner diameter at a first portion of the receiving ends 321 is different than the inner diameter at a second portion of the receiving ends 321. The outer diameter can be greater than the inner diameter. The middle-facing surface can extend between the outer and inner edges. Receiving ends 321 may have an inner diameter that is the same or less than a diameter of a corresponding coupling component (e.g., pins 309) to allow for a friction-fit connection. However, the cylindrical shape is not intended to be limiting. Receiving ends 321 may embody any shape, such as any shape that can receive coupling or alignment components (e.g., pins 309) of middle component 1105a and/or back component 1109a. In some aspects, receiving ends 321 may be tapered for a tight fit when receiving a coupling component. In some aspects, receiving ends 321 can include threads such that a coupling component can screw into the ends 321, or any other coupling mechanism described herein. Receiving ends 321 may fit or secure to pins 309.

Receiving ends 321 can be configured to secure via a friction force and/or a snap-fit, latch, and/or the like, to a coupling component of the middle component 1105a and/or the back component 1109a to prevent a shroud 201 from disassembly during use. Receiving ends 321 and pins such as pins 309 can help ensure that the cover component 1103a is aligned with the middle component 1105a and/or the back component 1109a when the cover component 1103a is secured to the middle component 1105a and/or the back component 1109a. Advantageously, receiving ends 321 can be used in substitution and/or in conjunction with other means of adhering one or more components of the shroud 201 (e.g., such as applying a glue between one or more components to provide a friction fit, securing the cover component 1103a to the middle component 1105a and/or the back component 1109a).

The middle component 1105a can include one or more features such as described with reference to FIG. 6E. For example, middle component 1105a can include an alignment component 311, loops 322, and receiving ends 323. In some embodiments, the middle component 1105a can include pins such as pins 318 shown and/or described in FIG. 6H

The shroud 201 can be configured with one or more features to ensure that the shroud 201 is secured and aligned with a transducer 213. For example, the middle component 1105a may include an alignment component such as alignment lip 311. The alignment lips 311 may be positioned on the middle portion such that the alignment lips 311 aligns with an alignment slot such as alignment slots 312 of the transducer. For example, the alignment lips 311 may be disposed at a middle portion of the middle component 1105a. In some examples, the alignment lips 311 may be disposed at or near an edge of the middle portion of middle component 1105a. The alignment lips 311 may extend along at least a portion of the middle portion, such as a portion of an edge of the middle portion. In some examples, the alignment lips 311 may extend away from the middle portion of middle component 1105a, such as outward laterally from an edge of the middle component 1105a. The alignment lip 311 may fit and/or secure to the alignment slot 312 of the transducer 213. For example, the alignment lip 311 may be polygonal in shape, such as trapezoidal. However, this is not intended to be limiting. The alignment lip 311 may embody any shape that can be received by alignment slot 312. The alignment lip 311 and the slot 312 can ensure that the transducer 213 and the shroud 201 are secured and remain in a fixed position while in use.

The loops 322 may be positioned on the middle component 1105a such that they align with corresponding pins 309 of the back component 1109a. For example, loops 322 may be disposed at an upper portion and/or bottom portion of the middle component 1105a. In some examples, the loops 322 may be disposed at or near a perimeter of the middle component 1105a. In some examples, the loops 322 may be disposed at or near and edge of the middle component 1105a, such as a top edge or side edge of the middle component 1105a. At least a portion of the loops 322 may extend away from the upper portion (and/or lower portion) of the middle component 1105a, such as extending laterally from an edge of the middle component 1105a. Loops 322 may be spaced from each other.

Loops 322 may be annular, ring-like, or cylindrical in shape. Loops 322 can have a cover-facing surface (e.g., facing toward the cover component when the middle component is secured to the cover component) and a back-facing surface (e.g., facing toward the back component when the middle component is secured to the back component). Loops 322 can include an outer sidewall extending between the cover-facing surface and the back-facing surface. The outer sidewall can include an outer edge having a constant or substantially constant inner diameter. In some aspects, the receiving ends 321 may be tapered such that the outer diameter at a first portion of the loops 322 is different than the outer diameter at a second portion of the loops 322. Loops 322 can include an inner sidewall extending between the cover-facing surface and the back-facing surface. The inner sidewall can include an inner edge having a constant or substantially constant inner diameter. In some aspects, the loops 322 may be tapered such that the inner diameter at a first portion of the loops 322 is different than the inner diameter at a second portion of the loops 322. The outer diameter can be greater than the inner diameter. The cover- and back-facing surfaces can extend between the outer and inner edges. The loops 322 may have an inner diameter that is the same as, or substantially the same as, a diameter of corresponding pins 309 to allow for a friction fit connection and/or to allow pins 309 to pass through loops 322. In some embodiments, the loops 322 may have an inner diameter that is larger or smaller (e.g., slightly larger or smaller) than a diameter of pins 309. In the example of FIGS. 6E-6H, the loops 322 are annular rings. However, this is not intended to be limiting. Loops 322 may embody any shape, such as any shape that can receive coupling or alignment components (e.g., pins 309) of back component 1109a and/or cover component 1103a.

Loops 322 can be configured to ensure that the back component 1109a is secured and aligned with the middle component 1105a, such as when the back component 1109a is secured to the middle component 1105a and/or to the cover component 1103a. The loops 322 can be configured to ensure that the pins 309 of the back component 1109 are aligned with the receiving ends 321 of the cover component 1103a, such as when the back component 1109a is secured to the cover component 1103a. Advantageously, loops 322 can be configured to reduce lift and angular moment of rotation of the back component 1109a when the back component 1109a is secured to the middle component 1105a and and/or secured to the cover component 1103a. Advantageously, loops 322 can be used in substitution and/or in conjunction with other means of adhering one or more components of the shroud 201 (e.g., such as applying a glue between one or more components to provide a friction fit, securing the back component 1109a to the middle component 1105a and/or the cover component 1103a).

Receiving ends 323 can be positioned on the middle component 1105a such that they align with a coupling or alignment component, such as securing posts 317 of the cover component 1103 shown and/or described in FIG. 6H. For example, the receiving ends 323 may be disposed at a top portion and/or middle portion of the middle component 1105a. In some examples, receiving ends 323 may be disposed at or near a perimeter of the middle component 1105a. In some examples, the receiving ends 323 may be disposed at or near an edge of the middle component 1105a, such as a top edge or side edge of middle component 1105a. At least a portion of the receiving ends 323 may extend away from the upper portion and/or middle portion of middle component 1105a, such as extending laterally from an edge of the middle component 1105a. In some aspects, the receiving ends 323 may extend along an axis aligned with that of one or more coupling or alignment components (e.g., securing posts 317) of the cover component 1103a and/or of the back component 1109a. Receiving ends 323 may be spaced from each other.

Receiving ends 323 may be hollow. In some aspects, receiving ends 323 may be cylindrical in shape. Receiving ends 323 can have a cover-facing surface (e.g., facing toward the cover component when the middle component is secured to the cover component) and a back-facing surface (e.g., facing toward the back component when the middle component is secured to the cover component). Receiving ends 323 can include an outer sidewall extending between the cover-facing surface and the back-facing surface. The outer sidewall can include an outer edge having a constant or substantially constant outer diameter. In some aspects, the receiving ends 323 may be tapered such that the outer diameter at a first portion of the receiving ends 323 is different than the outer diameter at a second portion of the receiving ends 323. Receiving ends 323 can include an inner sidewall extending between the cover-facing surface and the back-facing surface. The inner sidewall can include an inner edge having a constant or substantially constant inner diameter. In some aspects, the receiving ends 323 may be tapered such that the inner diameter at a first portion of the receiving ends 323 is different than the inner diameter at a second portion of the receiving ends 323. The outer diameter can be greater than the inner diameter. The cover- and back-facing surfaces can extend between the outer and inner edges. Receiving ends 323 may have an inner diameter that is the same or less than an outer diameter of a corresponding coupling component or alignment component (e.g., securing posts 317) to allow for a friction-fit connection. However, the cylindrical shape is not intended to be limiting. Receiving ends 323 may embody any shape, such as any shape that can receive coupling or alignment components (e.g., securing posts 317) of cover component 1103a and/or back component 1109a. In some aspects, receiving ends 323 may be tapered for a tight fit when receiving a coupling or alignment component. In some aspects, receiving ends 323 can include threads such that a coupling component can screw into the ends 323, or any other coupling mechanism described herein. Receiving ends 323 may fit or secure to securing posts, such as securing posts 317.

Receiving ends 323 can be configured to secure via a friction force and/or a snap-fit, latch, and/or the like, to a coupling or alignment component of the front component 1103a and/or the back component 1109a to prevent a shroud 201 from disassembly during use. Receiving ends 323 and securing posts such as securing posts 317 can help ensure that the cover component 1103a is aligned with the middle component 1105a and/or the back component 1109a when the cover component 1103a is secured to the middle component 1105a and/or the back component 1109a. Advantageously, receiving ends 323 can be used in substitution and/or in conjunction with other means of adhering one or more components of the shroud 201 (e.g., such as applying a glue between one or more components to provide a friction fit, securing the cover component 1103a to the middle component 1105a and/or the back component 1109a).

In some implementations, once secured, the locking features of shroud 201 may prevent separation of shroud components or opening of the shroud without forceful breaking of the shroud 201. In some implementations, one or more of the features mentioned herein (e.g., pins 309, coupling component 310, alignment lip 311, slot 312, coupling components 314, bottom groove 315, bottom lip 316, securing posts 317, pins 318, receiving ends 319, support posts 320, receiving ends 321, loops 322, receiving ends 323, and/or the like) may be used to secure one or more components of a shroud 201 (e.g., assemble, connect, and/or unite the back component 1109a, middle component 1105a, and/or cover component 1103a) in substitution or in addition to applying an adhesive substance (e.g., glue and/or the like) between the one or more components of the shroud 201.

FIG. 6G illustrates an exploded view of an additional implementation of the shroud 201. As illustrated, the shroud 201 may have a plurality of components, including but not limited to a back plate or back component 1109 configured to receive the transducer 213. The shroud 201 may have a middle component 1105 configured to secure the transducer 213 to within the shroud 201. The shroud 201 may include a cover plate or cover component 1103 configured to cover the top of the transducer 213. The cover component 1103 may include wings 305 further described in FIGS. 7A-7C below. In some examples, transducer wings 305 can include a texturized surface configured to provide a user with additional grip while securing the shroud 201 to mounting plates as described with further reference to FIGS. 7A-7C. The cover component 1103 may include a securing device 308 and/or a slot 313 further described in FIGS. 7D-7G.

The shroud 201 of FIG. 6G may include one or more features to ensure that a shroud 201 is secured and aligned with a transducer 213 as described with reference to FIGS. 6E-6F. For example, back component 1109 can include pins 309, a coupling component 310, a bottom lip 316, and support posts 320, such as described hereinabove with reference to FIGS. 6E-6F. Middle component 1105 may include an alignment component 311, loops 322, and receiving ends 323, such as described hereinabove with reference to FIGS. 6E-6F. In some embodiments, the middle component 1105 can include pins such as pins 318 shown and/or described in FIG. 6H. Cover component 1103 may include one or more coupling components 314, a bottom groove 315 such as described in FIGS. 6E-6F, one or more receiving ends 319 such as shown and/or described in FIGS. 6E-6F, and one or more receiving ends 321 such as shown and/or described in FIGS. 6E-6F. In some embodiments, the cover component 1103 can include one or more securing posts such as securing posts 317 shown and/or described in FIG. 6H

Coupling component(s) 314 may be disposed in the interior of the cover component 1103. The coupling component(s) 314 may be positioned on the interior portion such that they (it) align(s) with, for example, a receiving end of the middle component 1105. For example, the coupling component(s) 314 may be disposed at or near the perimeter of the interior portion of the cover component 1103. In some examples, the coupling component(s) 314 may be disposed at or near a top portion of the interior portion. In some examples, the coupling component(s) 314 may be disposed at or near an edge (e.g., a top edge or side edge) of the top portion. In some aspects, coupling components 314 may be spaced from each other, such as along an axis extending across the top portion of the interior of cover component 1103. For example, each coupling component 314 may be displaced from the center of the top portion of the interior portion.

In some aspects, the coupling component(s) 314 can include a snap-fit feature such as a snap hook or a mechanical fastener such as a latch. In some aspects, the coupling component(s) 314 can include threads and be screwed into a receiving end of the middle component 1105 and/or secured via a nut. The coupling component(s) 314 can include a fastener that secures the cover component 1103 to the middle component 1105 via a twisting motion (e.g., twist-and-lock). In some aspects, the coupling component(s) 314 can include interlocking tabs and/or grooves such as a ribs or barbs that grip the sides of a receiving end of middle component 1105. The coupling component(s) 314 may include magnets to secure the cover component 1103 to the middle component 1105 via a magnetic force.

The coupling component(s) 314 can provide a latch to secure the cover component 1103 to the middle component 1105 and/or the back component 1109. Coupling component(s) 314 can help ensure that the cover component 1103 is aligned with the middle component 1105 and/or the back component 1109 when the cover component 1103 is secured to the middle component 1105 and/or the back component 1109. Advantageously, coupling component(s) 314 can be used in substitution and/or in conjunction with other means of adhering one or more components of the shroud 201 (e.g., such as applying a glue between one or more components to provide a friction fit, securing the cover component 1103 to the middle component 1105 and/or the back component 1109).

In some implementations, once secured, the locking features of shroud 201 may prevent separation of shroud components or opening of the shroud without forceful breaking of the shroud 201. In some implementations, one or more of the features mentioned herein (e.g., pins 309, coupling component 310, alignment lip 311, slot 312, coupling component(s) 314, bottom groove 315, bottom lip 316, securing posts 317, pins 318, receiving ends 319, support posts 320, receiving ends 321, loops 322, receiving ends 323, and/or the like) may be used to secure one or more components of a shroud 201 (e.g., assemble, connect, and/or unite the back component 1109, middle component 1105, and/or cover component 1103) in substitution or in addition to applying an adhesive substance (e.g., glue and/or the like) between the one or more components of the shroud 201.

FIG. 6H illustrates an exploded view of an additional implementation of the shroud 201. As illustrated, the shroud 201 may have a plurality of components, including but not limited to a back plate or back component 1109 configured to receive the transducer 213. The shroud 201 may have a middle component 1105 configured to secure the transducer 213 to within the shroud 201. The shroud 201 may include a cover plate or cover component 1103 configured to cover the top of the transducer 213. The cover component 1103 may include wings 305 further described in FIGS. 7A-7C below. In some examples, transducer wings 305 can include a texturized surface configured to provide a user with additional grip while securing the shroud 201 to mounting plates as described with further reference to FIGS. 7A-7C. The cover component 1103 may include a securing device 308 and/or a slot 313 further described in FIGS. 7D-7G.

The shroud 201 of FIG. 6H may include one or more features to ensure that a shroud 201 is secured and aligned with a transducer 213 as described with reference to FIGS. 6E-6G. For example, back component 1109 can include pins such as pins 309 shown and/or described in FIGS. 6E-6F, a coupling component such as coupling component 310 shown and/or described in FIGS. 6E-6F, a bottom lip such as bottom lip 316 shown and/or described in FIGS. 6E-6F, and support posts such as support posts 320 shown and/or described in FIGS. 6E-6F.

Cover component 1103 may include one or more coupling components 314 such as shown and/or described in FIG. 6G, one or more securing posts 317, and one or more receiving ends 319 such as shown and/or described in in FIGS. 6E-6F. In some embodiments, the cover component 1103 can include a bottom groove such as bottom groove 315 shown and/or described in FIGS. 6E-6F.

Securing posts 317 may be disposed in the interior portion of the cover component 1103. The posts 317 can be positioned on the interior portion such that they align with corresponding receiving ends 323 of middle component 1105. For example, posts 317 may be disposed at or near the perimeter of the interior portion of the cover component 1103. In some examples, posts 317 may be disposed at or near an edge of the inner portion. In some examples, posts 317 may be disposed at a top portion and/or middle portion of the interior portion of cover component 1103. Posts 317 may extend from the interior portion of cover component 1103. In some aspects, the posts 317 may extend along an axis aligned with that of one or more receiving ends 323 of the middle component 1105. In some aspects, posts 317 may be spaced from each other, for example, such that posts 317 may be equidistant from each other.

Securing posts 317 may be solid or hollow. Posts 317 may be cylindrical in shape and may have the same diameter as, or a slightly larger diameter than, an inner diameter of the receiving end 323 to allow for a friction-fit connection. Securing posts 317 may have the same diameter or a different diameter than those of pins 309 or posts 320. For example, posts 317 may be larger in diameter than are pins 309. In some examples, posts 317 may be smaller in diameter than are posts 320. However, the cylindrical shape is not intended to be limiting. Posts 317 may embody any shape, such as any shape that can be received within receiving ends 323 of the middle component 1105. In some aspects, posts 317 may be tapered for a tight fit when received within the receiving ends 323. In some aspects, the posts 317 may include threads and can be screwed into receiving ends 323 of the middle component 1105 and/or or secured with a nut. In some aspects, securing posts 317 can include a snap hook (e.g., snap-fit) mechanism, a latch, or interlocking tabs and/or grooves such as a ribbed or barbed shaft that grips the sides of the receiving ends 323. In some aspects, at least a portion of the shafts of posts 317 may be magnetic to secure to the receiving ends 323 via a magnetic force.

Securing posts 317 can be configured to secure via a friction force and/or a snap-fit, latch, and/or the like, to a receiving end 323 of the middle component 1105 and/or the back component 1109 to prevent a shroud 201 from disassembly during use. Securing posts 317 can help ensure that the cover component 1103 is aligned with the middle component 1105 and/or back component 1109 when the cover component 1103 and middle component 1105 are secured to one another. Advantageously, securing posts 317 can be configured to reduce lift and angular moment of rotation of the cover component 1103 when the cover component 1103 is secured to the middle component 1105 and wings 305 are compressed. Advantageously, posts 317 can be used in substitution and/or in conjunction with other means of adhering one or more components of the shroud 201 (e.g., such as applying a glue between one or more components to provide a friction fit, securing the back component 1109 to the middle component 1105 and/or the cover component 1103).

Middle component 1105 can include an alignment component 311 such as shown and/or described in FIGS. 6E-6F, loops 322 such as shown and/or described in FIGS. 6E-6F, receiving ends 323 such as shown and/or described in FIGS. 6E-6F, and pins 318.

Pins 318 may be disposed in the interior portion or exterior portion of the middle component 1105. The pins 318 can be positioned on the interior portion or exterior portion such that they align with corresponding receiving ends 319 of cover component 1103. For example, pins 318 may be disposed in a bottom portion of middle component 1105. In some examples, pins 318 may be disposed at or near the perimeter of a bottom portion of the middle component 1105. In some examples, pins 318 may be disposed at or near an edge of the bottom portion, such as a bottom edge of middle component 1105. The pins 318 can be spaced from each other. Pins 318 may extend from the middle component 1105. In some aspects, the pins 318 may extend along an axis aligned with that of one or more receiving ends 319 of the cover component 1103. In some aspects, the pins 318 may extend from the bottom portion of the middle component 1105 toward the back component 1109. For example, the pins 318 may extend along an axis different from the axis along which the bottom edge of the bottom portion extends. The pins 318 may extend along an axis that is perpendicular to the axis along which the bottom edge of the bottom portion extends.

Pins 318 may be solid or hollow. Pins 318 may be cylindrical in shape and may have the same diameter as, or a slightly larger diameter than, an inner diameter of the receiving ends 319 to allow for a friction-fit connection. However, the cylindrical shape is not intended to be limiting. Pins 318 may embody any shape, such as any shape that can be received within receiving ends 319 of the cover component 1103. In some aspects, pins 318 may be tapered for a tight fit when received within the receiving ends 319. In some aspects, the pins 318 may include threads and can be screwed into receiving ends 319 of the cover component 1103 and/or or secured with a nut. In some aspects, pins 318 can include a snap hook (e.g., snap-fit) mechanism, a latch, or interlocking tabs and/or grooves such as a ribbed or barbed shaft that grips the sides of the receiving ends 319. In some aspects, at least a portion of the shafts of pins 318 may be magnetic to secure to the receiving ends 319 via a magnetic force. The pins 318 may provide a protrusion such as an alignment ridge or rail that can be received by a groove or slot (e.g., an alignment slot) of receiving ends 319. For example, pins 318 may fit or secure to receiving ends 319.

Pins 318 can be configured to secure via a friction force and/or a snap-fit, latch, and/or the like, to a receiving end 319 of the cover component 1103 to prevent a shroud 201 from disassembly during use. Pins 318 and receiving ends 319 can help ensure that the middle component 1105 is aligned with the cover component 1103 and/or the back component 1109 when the middle component 1105 is secured to the cover component 1103 and/or the back component 1109. Advantageously, pins 318 can be used in substitution and/or in conjunction with other means of adhering one or more components of the shroud 201 (e.g., such as applying a glue between one or more components to provide a friction fit, securing the middle component 1105 to the cover component 1103 and/or the back component 1109).

In some implementations, once secured, the locking features of shroud 201 may prevent separation of shroud components or opening of the shroud without forceful breaking of the shroud 201. In some implementations, one or more of the features mentioned herein (e.g., pins 309, coupling component 310, alignment lip 311, slot 312, coupling component 314, bottom groove 315, bottom lip 316, securing posts 317, pins 318, receiving ends 319, support posts 320, receiving ends 321, loops 322, receiving ends 323, and/or the like) may be used to secure a shroud 201 in substitution for applying an adhesive substance (e.g., glue and/or the like).

As shown in FIGS. 7A-7C, a transducer shroud 201 can include one or more movable wings 305. The wings 305 may be configured to engage with a mounting plate 301 through brackets 303. The mounting plate 301 and brackets 303 may be any number of shapes and/or configurations. One or more of the movable wings 305 may be configured to provide an outward force when moved or deformed towards a center of the transducer shroud 201. The outward force may provide sufficient outward tension or force to removably couple the transducer shroud 201 to the mounting plate 301. FIG. 7A illustrates an example top view of a transducer shroud 201 mounted on a mounting plate 301. As illustrated in FIG. 7A, the wings 305 may be configured to engage with brackets 303 of a mounting plate 301. The brackets 303 may be any number of sizes and/or distances from each other on the mounting plate 301. Advantageously, the wings 305 may be configured to couple the transducer shroud 201 to the mounting plate 301 with brackets 303 that are configured to be of different distances from each other, different shapes, different sizes, and/or the like. Accordingly, the transducer shroud 201 may be mounted to a variety of different types of mounting plates 301 with different bracket 303 orientations. In some examples, the transducer shroud 201 may be configured to mount to brackets 303 having a distance between the first and second bracket of approximately 30 mm, 28 mm, 26 mm, and/or any distance between, or greater than or less than 30 mm or 26 mm. In some examples, the distance between brackets 303 may be referred to as a slot. A depth of the slot may be any number of depths, including, but not limited to approximately 2.5 to 5.5 mm and/or a value outside of that range. The transducer shroud 201 can include a slot 211 (e.g., a receptacle and/or the like). Slot 211 can be configured to secure a transducer shroud 201 to a medical IV pole (e.g., medical IV pole 206 of FIG. 2) and/or the like. In some implementations, wings 305 can include a texturized surface. The texturized surface can provide a user with additional grip while securing the shroud 201 to mounting plates 301.

FIGS. 7B and 7C illustrate perspective views of example shroud 201a, 201b having wings 305a and 305b, respectively. The wings 305a, 305b may facilitate universal and/or near universal mounting to a plate 301. For example, the wings 305a, 305b may be integrated and flex with a large enough range so as to mount the shroud 201a, 201b, to mounting plates 301 with small and large distances between mounting brackets 303. As illustrated, a transducer shroud 201a, 201b may have different shapes, including, but not limited to a curved shape, such as illustrated in FIG. 7B, or a substantially flat shape, such as illustrated in FIG. 7C. The shape of the wing 305a, 305b may be similar to the shape of the transducer shroud 201a, 201b. For example, in a curved shape example, a flex arm or wing 305a may be curved. In another example, a flex arm or wing 305b may be substantially flat or a hinged petal. The flex arm or wing 305a, 305b may be part of a molded shape of the transducer shroud 201a, 201b, such as illustrated in FIGS. 6A-6H. For example, the shroud 201a, 201b may include a single unit of rigid or semi-rigid material on at least a side portion of the transducer 213. A wing 305a and/or 306b may, in some examples, form a part of that single unit with cutouts to allow for movement of the wing 305a and/or 306b from a main part of the single unit of material. In some examples, the wing 305a and/or 306b may be a separate component. For example, the wing 305a and/or 305b may be a separate component coupled to a spring system to allow for movement of the wing 305a and/or 305b in coupling the transducer shroud 201a, 201b (and/or a transducer 213) to a mounting plate 301.

FIGS. 7D-7G illustrate perspective views an example shroud 201c including wings 305c, a slot 313, and/or a securing device 308. Wings 305c and/or a securing device 308 can be removably coupled to the bracket 303. A securing device 308 and/or slot 313 can engage the bracket 303 and/or mounting plate 301 to prevent the shroud 201c from unwanted movement and/or sliding along brackets 303 and/or the mounting plate 301. When engaged, the securing device 308 and/or slot 313 can act as a physical stop, preventing movement of the shroud 201c along the bracket 303 and/or mounting plate 301.

A securing device 308 can extend from a wing 305c into brackets 303 as depicted in FIGS. 7D-7G. In some examples, a securing device 308 can be located on an exterior portion of wings 305c. Additionally and/or alternatively, a securing device 308 be positioned at another location along the wing 305c (e.g., an edge, back, top, bottom, interior, along the middle, and/or the like). As depicted in FIGS. 7D-7G, a securing device 308 can be a hemispherical and/or another geometric shape (e.g., dome shape and/or the like), protruding outward form an exterior portion of a wing 305c. The securing device 308 can be rigidly flexible, to create a spring-like tension when engaged between the shroud 201c and brackets 303. Additionally and/or alternatively, a securing device 308 may have another shape (e.g., an indent, knob, protrusion, fastener, latch, point, an asymmetric hemispherical shape, and/or the like).

The securing device 308 can facilitate an outward force against a bracket 303, to support various shapes, sizes, and weights of a shroud 201c and/or provide stability while a shroud 201c is mounted in brackets 303. For example, a securing device 308 may prevent movement of a shroud 201c while a patient moves (e.g., tension created by a physical link from one or more physiological sensors connected between a patient and the shroud 201c, as a patient moves about). In some examples, the securing device 308 may temporarily prevent movement of the shroud 201c, allowing a user to de-couple the securing device 308 (e.g., the shroud 201c) from brackets 303 when a new patient and/or new location for the shroud 201 is desired, such as moving from room to room in a hospital and/or moving the shroud 201c from a first mounting plate to a second mounting plate along a hospital bed.

In some examples, a securing device 308 can provide tactile feedback (e.g., haptic feedback and/or the like) to a user while attaching a shroud 201c to mounting brackets 303. For example, as a shroud 201c is placed in brackets 303, the securing device 308 may engage a mounting bracket 303 to produce a vibration throughout the shroud 201a. The vibration traveling through a shroud 201c may be received by a user while holding the shroud 201c. Advantageously, the vibration can indicate to the user, that the shroud 201c is properly secured to the mounting brackets 303. Additionally and/or alternatively, a securing device 308 can provide audible feedback to a user while attaching a shroud 201c to mounting brackets 303. For example, as a shroud 201c is placed in brackets 303, the securing device 308 may engage a mounting bracket 303 to produce a vibration throughout the shroud 201c. The vibration traveling through a shroud 201c may generate an audible signal to a user while the user is positioning the shroud 201c in the mounting brackets 303. Advantageously, the audible signal can indicate to the user, that the shroud 201c is properly secured to the mounting brackets 303.

The wing 305c can further include a slot 313. The slot 313 can be configured to facilitate an inward and/or outward movement of a securing device 308. In some examples, a slot 313 can facilitate movement of the securing device 308 independent of the wing 305c. A slot 313 can enable the shroud 201c to rest on and/or lock to the mounting bracket 303. A shroud 201c can include one, two, three and/or more securing devices 308. In some examples, a shroud 201c can include one or more securing device 308 for each wing 305c (e.g., one securing device 308 per each wing 305c and/or the like). Additionally and/or alternatively, a slot 313 can be located adjacent to a mounting device 308. In some examples, a slot 313 can be included in another location along wings 305c.

The wings 305c and/or securing device 308 may be integrated and flex with sufficient range to mount the shroud 201c to a variety of mounting plates 301, such as mounting plates 301 having small and large distances between mounting brackets 303. The wings 305c and/or securing device 308 may be part of a molded shape of the transducer shroud 201c, such as illustrated in FIGS. 7D-7G. For example, the shroud 201c may include a single unit of rigid or semi-rigid material on at least a side portion of the transducer 213.

A wing 305c and/or securing device 308 may, in some examples, form a part of a single unit with cutouts to allow for movement of the wing 305c and/or securing device 308 from a main part of the single unit. In some examples, a wing 305c and/or securing device 308 may be a separate component and secured to the shroud 201c. Additionally and/or alternatively, wings 305c, a securing device 308, and/or a slot 313 may be a separate component coupled to a spring system to allow for movement of the securing device 308 in coupling the transducer 213 to a mounting plate 301 of FIG. 7A.

In some implementations, wings 305c can optionally include a texturized surface. The texturized surface can provide a user with additional grip while securing the shroud 201c to mounting plates 301. In some examples, a texturized surface can be located on the exterior portion of wings 305c. The texturized surface can include one or more different surfaces added to and/or molded into wings 305c. For example, a texturized surface can include one or more of bumps, a rubberized surface, an abrasive texture, varying depths, one or more patterns and/or designs, and/or any other surface to improve grip, traction, and/or the like.

In addition and/or alternative to a texturized surface located on wings 305c, the texturized surface can be located on an exterior surface of one or more components of a shroud 201c such as a cover component 1103c, middle component 1105c, and/or back component 1109c. Additionally and/or alternatively, a texturized surface may be located on an exterior surface of wings 305, 305a, and/or 305b, and/or components associated with shroud 201, 201a, and/or 201b as disclosed herein.

### Example Aspects Related to Graphical User Interfaces

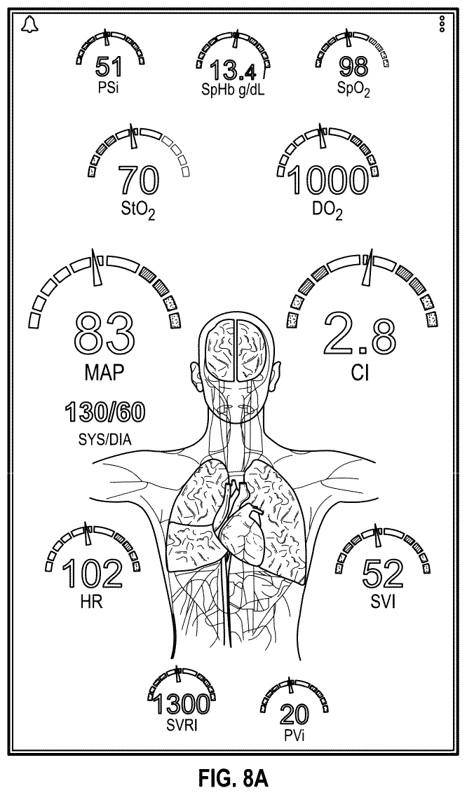

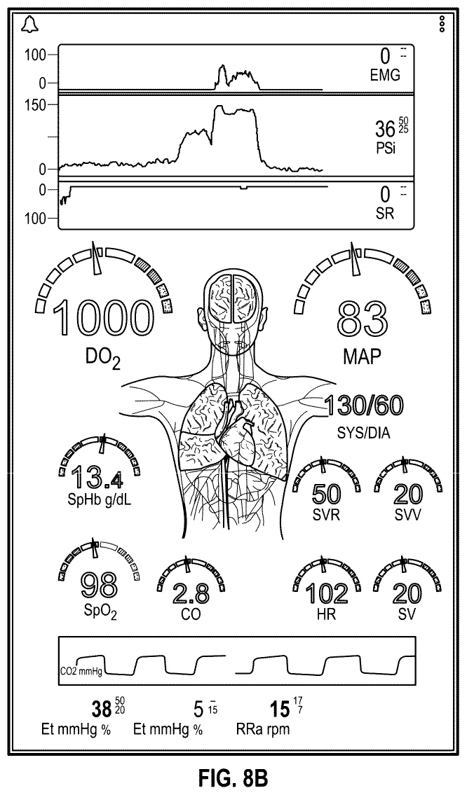

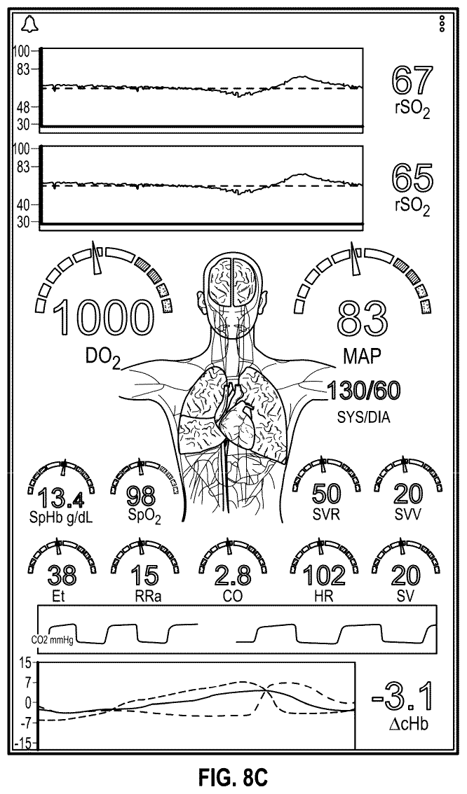

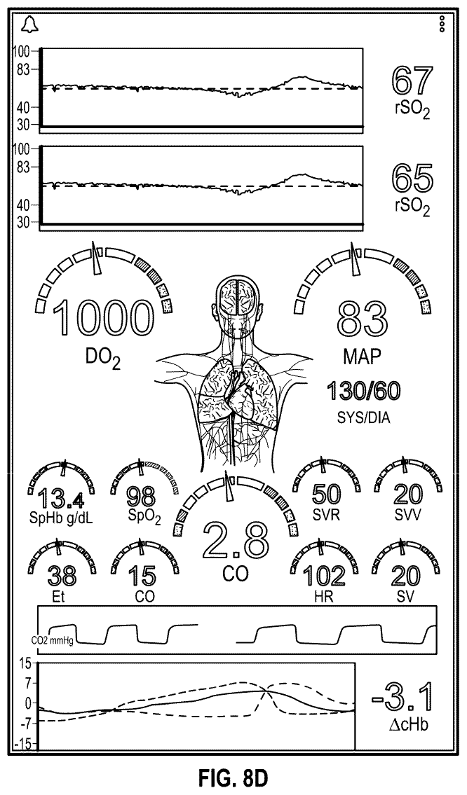

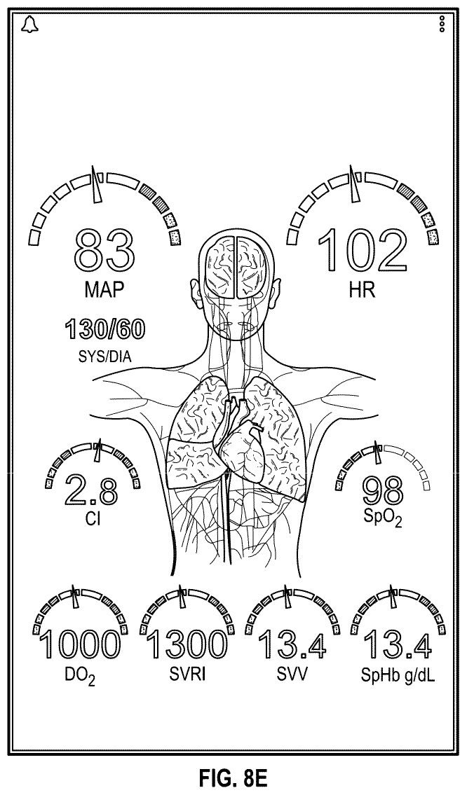

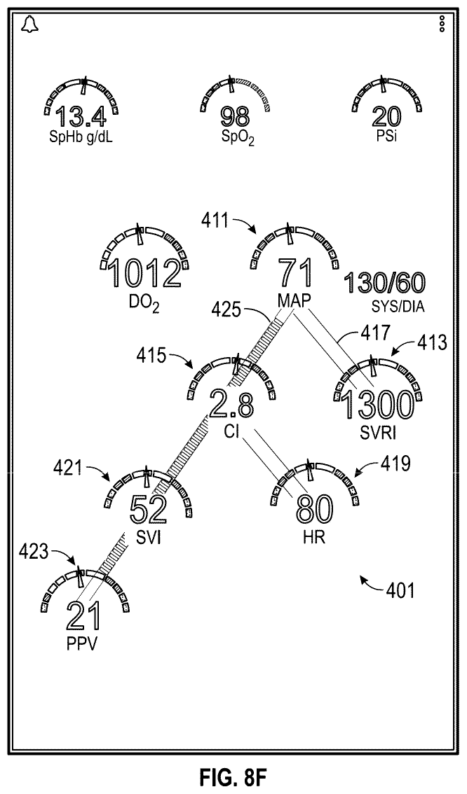

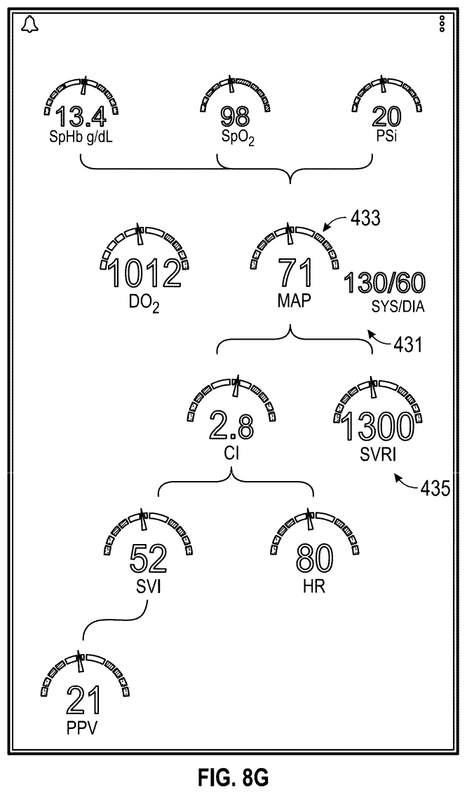

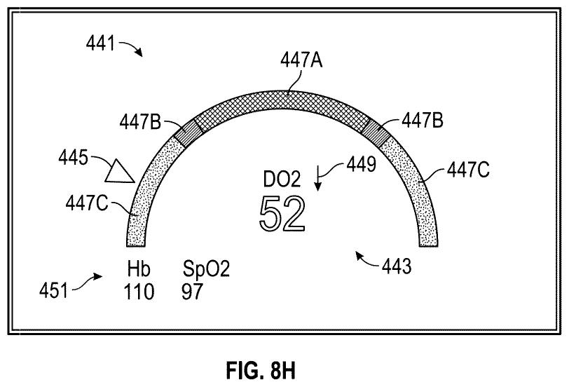

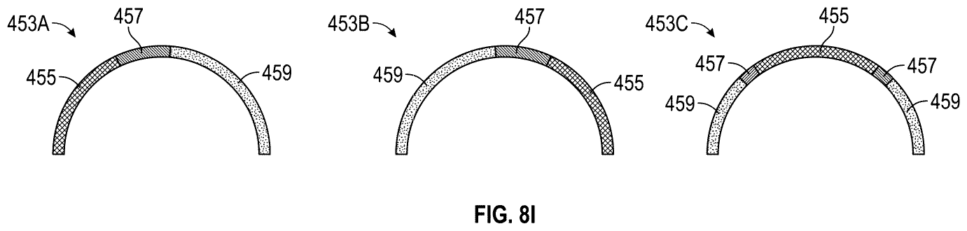

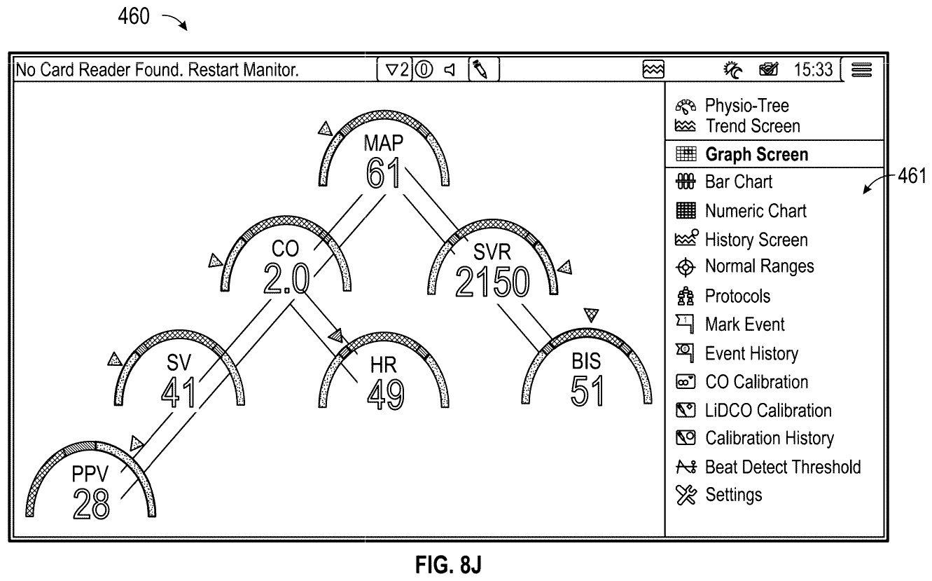

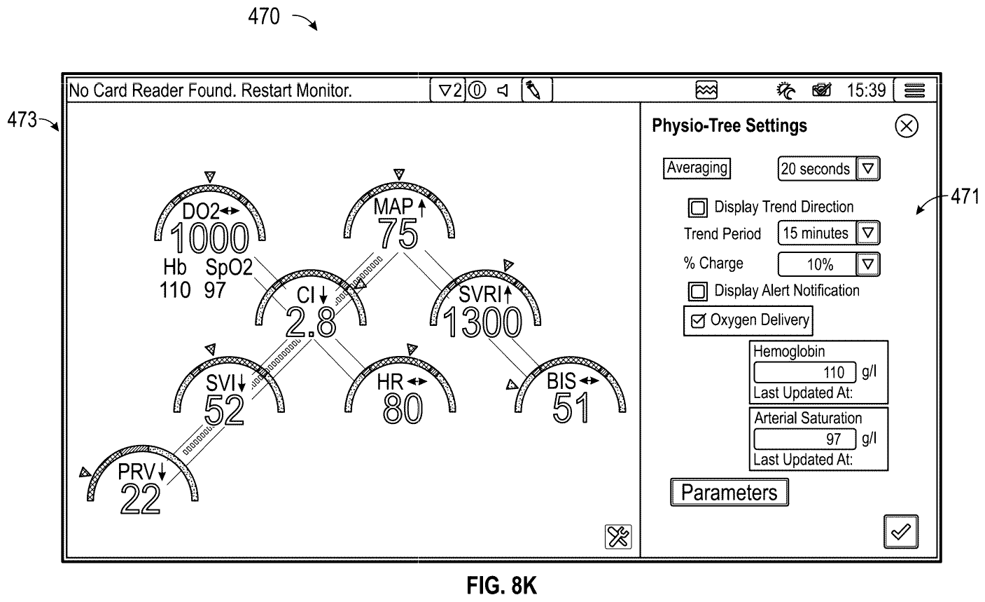

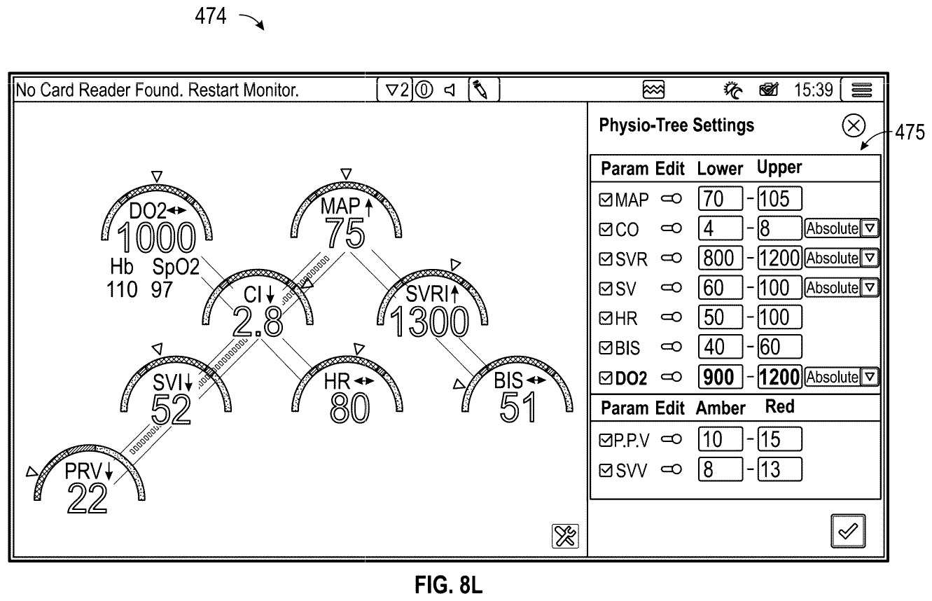

FIGS. 8A-8G, 9A-9G, 10A-10D, 11A-11D, 12A-12C, 13A-13F illustrate example user interfaces that may be displayed by a monitor device of the system described herein, such as example monitor device 105, 105a described with reference to FIG. 1 and/or example monitor devices 205a or 205b described with reference to FIG. 2. The data for rendering the graphical user interfaces described below may be generated by an adapter of the system described herein, such as example adapter 103 described with reference to FIG. 1 and/or example adapter 203 described with reference to FIG. 2.

FIGS. 8A-8G show example user interfaces that may display parameters or information relating to a patient's hemodynamic status and/or other physiological parameters or information. The user interfaces of FIGS. 8A-8G may be interactive and may be configured to receive a user input. For example, a user may touch the display at various portions of the user interface which may cause the user interface to update, display different data, display a different user interface and/or the like.

FIGS. 8A-8E illustrate aspects of an example interface that may include at least one graphical representation of physiological conditions of a patient. The graphical representation may include a graphical representation of a patient's physiology. In some examples, aspects of the physiology may be accented or highlighted, such as the lungs or the brain in the graphical representation, based on physiological parameters associated with the patient. For example, a color, brightness, or other aspect of the representation of the physiology may be changed based on a physiological parameter. For example, if a patient's physiological parameters indicate healthy lung function, the lungs of the patient may be shown as green or another color. If the patient's physiological parameters indicate abnormal lung function, the lungs of the patient may be shown as a different color than green. In some examples, the graphical representation of the patient's physiology may be emphasized or changed based on the selected physiological parameters to display. For example, if SpO2 is selected for display, a patient's lung function or other parameter associated physiology may be graphically represented or highlighted. A size of the graphical representation may be changed based on a selected layout of parameters.

In some examples, different types and/or aspects of a graphical representation may be emphasized or shown based on a user selection. For example, one or more of a graphical representation of a patient physiology, radial gauge associated with a physiological parameter value, textual representation of a physiological parameter value, graphical or waveform representation of a physiological parameter value, or other aspect or representation of a measured physiological parameter may be displayed on a monitor. The size, location, and presence of one or more of the aforementioned representations may be adjusted based on a user input. FIGS. 8A-8E illustrate example screens with alternative orientations and selections for display of physiological parameters.

FIGS. 8F-8G illustrate some example interfaces for display of relationships between physiological parameters. For example, as illustrated in FIG. 8F, a GUI may be configured to display one or more relationships in a tree or factorial structure. Advantageously, the configuration of the interface may facilitate easy and fast understanding of the relationship between parameters in addition to the value of parameters themselves. For example, the layout, order, and/or emphasis of physiological parameters may help transmit information to the user of the interface.

FIG. 8F illustrates an example layout of parameters in a hierarchical tree 401. The hierarchical tree 401 may include a plurality of related parameters associated with one or more physiological parameters. For example, a blood pressure associated score (such as a mean arterial pressure or MAP score) 411 may be related to CO (CI), DO2 (DO2I), SVR (SVRI), SV, HR, PPV, BIS, dPdt, EA-dyn. The interface may be configured to show a mathematical or influencing relationship between parameters by arranging the parameters in a hierarchy (such as a pyramid).

The level at which a parameter is displayed in the hierarchy may be associated with the influencing relationship of the parameter with the at least one main or primary parameter. For example, the interface may display at least one main or primary parameter on a first level and influencing parameters (or parameters that may influence the at least one main or primary parameter) on lower levels. In some examples, the level below a main or primary parameter at which an influencing parameter is displayed may be associated with the relative importance of the influencing parameter on the main or primary parameter. In some examples, the level below a main or primary parameter may be associated with the relative dependence of the influencing parameter on the main or primary parameter. Other associations may additionally or alternatively be used for determining a location for display of a parameter in the hierarchy tree 401. In some examples, the interface may display at least one main or primary parameter at the top of a geometry and at least one first influencing parameter immediately below the at least one main or primary parameter. Parameters that influence the at least one first influencing parameter may be displayed below the at least one primary or main parameter, such as a mean arterial pressure, and lower areas of the geometry are populated by parameters that influence the main parameter. For example, the MAP 411 may be influenced by CO 415 and SVR 413. Thus, CO 415 and SVR 413 may be displayed below the MAP 411 in the tree 401. In another example, the CO 415 may be influenced by SV 421 and HR 419. Thus, SV 421 and HR 419 may be displayed below the CO 415 in the tree 401. In another example, SVR 413 may be influenced by BIS. Thus, BIS may be displayed below SVR 413. In another example, SV 421 may be influenced by PPV (or SVV) 423. Thus, PPV (or SVV) 423 may be displayed below SV 421. Other parameters may additionally or alternatively be displayed in the same or other positions.

Relationships between parameters may additionally or alternatively be displayed using lines, connectors, or other graphics configured to indicate a connection between a first and second parameter. FIG. 8F illustrates at least one graphic between parameters in the shape of a line or dashed line configured to indicate a connection between two or more parameters. FIG. 8G illustrates an alternative method indicating relationships between parameters. For example, one or more brackets 431 may be used to indicate that one or more influencing parameters 435 are related to one or more other parameters 433. Other types of graphics may additionally or alternatively be used.

In some cases, relationships between parameters may be highlighted or otherwise accented based on whether an influencing parameter 435 is currently influencing or more strongly influencing a value or trend of a particular parameter. For example, as is illustrated in FIG. 8F, the relationship line 425 between CI and MAP is accented or highlighted by a dashed line in comparison to the relationship line 417 between MAP and SVRI. The relationship line may be accented when a property or value of the influencing parameter 435 is contributing to the current value of the main parameter. The accenting may be implemented as an alert for different scenarios. The alert may be turned on or off based on a user input. Advantageously, this can allow a caregiver to quickly perceive the source of an issue and respond to a patient with a new treatment regimen if necessary or understand if applied treatments are working. Table 1 illustrates some example clinical scenarios where MAP relationships between parameters may be accented. It is of note that merely using MAP as an indicator for diagnosing hemodynamic conditions can result in inaccurate diagnoses. Thus, having multiple parameters contributing to the MAP and what is influencing the MAP may be useful in a clinical setting for improved medical care.

TABLE 1
Displaying different clinical scenarios for the state of influencing
parameters on MAP scores that may be indicated by a hierarchical
tree and relationship display such as described herein.

| Scenario | MAP | CO | SVR | HR | SV | SVV |
|---|---|---|---|---|---|---|
| Aesthetic | Low | | Low | | | |
| Induced | | | | | | |
| Hypotension | | | | | | |
| Sepsis induced | Low | High | Low | High | Low | High |
| Hypotension | | | | | | |
| Fluid | Normal/ | | | | Low | High |
| Responsive | Low | | | | | |
| Shock State - | Low | High | | | | High |
| Hypovolemic | | | | | | |
| Shock State - | Low | Low | High | | | Normal/ |
| Cardiogenic | | | | | | Low |
| Shock State - | Low | Normal/ | Low | | | High |
| Neurogenic | | Low | | | | |
| Shock State - | High/ | Low | | | | High |
| Anaphylactic | Normal | | | | | |
| Shock State - | Low | High | Low | High | | High |
| Septic (early) | | | | | | |
| Shock State - | Low | Low | High | Low | | Low |
| Septic (late) | | | | | | |
| Shock State - | Low | Low | High | | | |
| Obstructive | | | | | | |

Parameter values may be indicated in one or more ways on the interface. For example, a parameter value may be indicated by display of the numerical value, an approximation of the numerical value, an index associated with a numerical value, or another alphanumeric representation of the parameter value. Parameter values may be indicated through a graphical representation of the parameter value. For example, a parameter value may be displayed as or in association with a radial gauge. The radial gauge may be a graphic, such as an image updated at intervals or an animation. FIG. 8H illustrates example radial gauge 441 that may be utilized as part of an interface. For purposes of clarity in the figures, only one of the radial gauge 441 displays are indicated. However, each of the parameters 443, such as SVI, are shown to have a corresponding radial gauge 441 display. In some examples, some or all of the displayed parameters may have an associated radial gauge 441. In the illustrated example, the radial gauge 441 may be configured to indicate a value of the parameter in relation to a reference value and/or range of values. A radial gauge 441 may have a plurality of segments 447A, 447B, 447B and/or an indicator 445. The indicator 445 may be an arrow, needle or other image or animation configured to move and/or otherwise indicate a location of a value in relation to the segments 447A, 447B, 447C. The segments 447A, 447B, 447C may indicate ranges or zones in which a parameter value is normal or abnormal. For example, a segment 447A may indicate a normal or healthy range of values for a parameter. One or more segments 447B may indicate warning or slightly abnormal values for a parameter. If an indicator 445 is illustrated as pointing or near a particular segment, the parameter may be within those range of values associated with that particular segment. Additionally or alternatively to the radial gauge 441, a graphic 449 may be displayed to indicate information associated with the parameter being illustrated. For example, a graphic 449 may include an arrow, emoji, or other graphic to indicate a trend, health, accuracy, or other information associated with a parameter value. In the illustrated example, a graphic 449 includes an arrow. The direction of the arrow may correspond to a rate of the trend. For example, an arrow pointing down may indicate a downward trend and an arrow pointing up may indicate an upward trend. The arrow is shown as pointing down in order to indicate a downward trend of the parameter value. The arrow may have a color configured to illustrate a current status of the value. For example, a red arrow may indicate that the value is within an unhealthy range of values, a yellow arrow may indicate that the value is within a warning range of values, and a green arrow may indicate that the value is within a healthy range of values. In some examples, one or more associated parameters 451 may be displayed at or near the radial gauge 441 and/or parameter 443.

One or more segments 447C may indicate highly abnormal, cautionary, or otherwise bad range of values for a parameter. FIGS. 81-8J illustrate details of how segments may be illustrated on a radial gauge. For example, a radial gauge may have a plurality of colors. Each segment may have an associated color. The colors may be contrasting for case of readability. In some examples, the colors of the segments may be shaded differently based on whether a value is within a range associated with the present segment. For example, FIG. 8I illustrates a plurality of different types of radial gauge components 453A, 453B, 453C. Radial gauge component 453A illustrates an example single sided radial gauge segment orientation wherein a lower range is normal. Radial gauge component 453B illustrates an example single sided radial gauge segment orientation wherein an upper range is normal. Radial gauge component 435C illustrates an example double sided set of limits where a central range is normal and lower or higher ranges are both abnormal. A radial gauge component (e.g., 435A, 435B, and/or 435C) type may be based on the type of parameter. For example, a radial gauge component 435A associated with a normal lower range may be associated with PPV and SVV and/or a radial gauge component 435C associated with a normal central range may be associated with HR, MAP, SV, CO, DO2, SVR, and BIS. Other radial gauge component types and other parameter associations are also possible.

A layout of the radial gauge segments for a particular radial gauge component may be fixed for a particular parameter. For example, a radial gauge segment may have a fixed size and position. A value for ranges associated with one or more of the segments may be normalized or otherwise selected so that the interface is consistent and easy to read. An overall range may change from parameter to parameter. For example, a distribution of range among segments may include: 40% for normal segments, 5-15% for abnormal segments and 35-45% fir abnormal segments. Other distributions may also be possible, and/or distributions may be dependent on radial gauge type and layout.

In the illustrated examples, a normal range is associated with segment 455. Segment 455 may be shaded green or other permissive color. Further, a warning range is associated with segment 457 that is slightly outside the range associated with segment 455 (such as above or below segment 455). Segment 457 may be shaded a different color from segment 455, such as a yellow or warning associated color. Further, an abnormal or danger range is associated with segment 459 that is outside the range associated with segment(s) 457. Segment 459 may be shaded a different color from segments 455 and 457, such as a red or non-permissive color. When a value of the parameter or indicator of the value is within a particular segment or associated segment range, such as segment 455, a color or other aspect of the segment may change. For example, segment 455 may be accented, such as by being brightened in color when the value falls within that range.

An interface may display a trend status. As illustrated in FIG. 8H, a graphic 449 may be indicated near a parameter 443 or radial gauge 441. A trend status may depict a direction of a parameter trend over a course of time relative to a baseline taken at a prior time. The baseline and/or time range may be user adjustable, such as a minute to 10-minute trend over a baseline of 30 seconds prior to the start of the time period. A baseline value may be an average of all data above a certain confidence level over a determined time period prior to a trend period. For example, a baseline value may be a 30 second average of all good beat data prior to a trend period. The baseline value may be considered valid for use if the ratio of good data is greater than or equal to 70% or another percentage. Good data may include data that is over a determined confidence level. A trend period may include 5, 15, 30, or 60 minutes. The trend period may be adjustable in 5-minute increments. The longer the trend period, the slower to change the trend indicator. A trend calculation may be a sample of 30 seconds of data at the end of a trend period over the baseline value. The baseline value may be considered valid for use if the ratio of good data is greater than or equal to 70% or another percentage. A trend calculation may be updated at intervals or beat to beat. In some examples, a trend calculation may be updated at intervals that is not beat to beat in order to reduce jitter. A trend direction may be determined based on a range. For example, an upward trend may be associated with a greater than or equal to a limit percentage (for example, 10%, 15%, or 20%) increase from baseline. In another example, a downward trend may be associated with a greater than or equal to a limit percentage (for example, 10%, 15%, or 20%) decrease from baseline. In another example, a stable trend may be associated with a range less than or equal to the determined limit percentage for the upward or downward trend. In some examples, the stable trend may be associated with a range, of for example, between −8% and +8% and the upward and/or downward limit percentage may be 10%. A gap between the limit percentages and the stable range (such as 2% or other) may be used in order to reduce sudden trend changes. Accordingly, a trend indicator may be maintained until a new condition occurs.

A trend status may convey not only direction of the trend, such as an increase or decrease or stability of the trend, but also the zone that the trend is heading towards, such as normal or abnormal. The trend status may facilitate eased understanding of a patient's physiological condition at a glance. In some examples, a trend status color may be determined by a zone or range of values in which the current parameter value lies. A green trend status indicator may indicate a trend direction (either positive or negative) while in a stable, healthy, normal, or green zone. A red status indicator may indicate a trend direction while outside of a stable, healthy, normal, and/or green zone. A grey or hollow status indicator may indicate a trend direction regardless of location of the parameter, the trend is stable. For example, a parameter may be abnormal currently, but trending towards normal. The trend status would thus indicate trending upwards towards normal. In another example, a parameter may be currently normal but trending towards abnormal. The trend status would thus indicate trending towards abnormal. In some examples, a color of a trend status may indicate a rate of a trend. In some examples, a color may indicate the current range of the parameter while a shape or orientation of an arrow associated with the trend status indicates the trend. For example, a trend status indicator can include a single sided or double-sided arrow. An up arrow may indicate trending towards an upward direction. A downward arrow may indicate trending towards a downward direction. A double-sided flat arrow may indicate a stable parameter or no or flat trend. A size of a trend may be an aspect of the graphic associated with the trend status, for example, a size of trend may be indicated by color, angle of the arrow, size of the arrow, or a combination thereof. An interface may include a plurality of settings and/or display options that may be accessed through a menu. FIG. 8J illustrates an example menu 461 that may be part of an interface 460, such as illustrated in FIG. 8F, that may be used to access different aspects of the interface, display options, and/or update or personalize display settings. Aspects of the interface may include a physio-tree (or hierarchy display such as described above). The interface may additionally or alternatively include a trend screen, graph screen, bar chart, numeric chart, history screen, normal ranges, protocols, mark event, event history, CO calibration, LiDCO calibration, calibration history, beat detect threshold, and settings. The menu 461 may be accessed through a menu icon. In some examples, the interface may be configured to change color theme based on a user selection of a theme setting, such as a day or night theme via the menu 461.

FIG. 8K illustrates an example settings screen 471 of an interface 470. In the illustrated example, the interface 470 may allow a user to update settings of the physio-tree or hierarchy layout 473. Settings may include but are not limited to: a selection for averaging time, a selection for whether to display a trend direction, a setting for trend period, a setting for percentage limit or percentage change for the trend, a setting to display alert notifications, settings for displaying of oxygen delivery related parameters, and/or settings for updating the parameters to display.

FIG. 8L illustrates an example parameter setting screen 475 of an interface 474 in which parameters may be selected for display. In some examples, the parameter setting screens 475 may allow a user to select one or more parameters for display, including but not limited to MAP, CO, SVR, SV, HR, BIS, DO2, PPV, and SVV. The parameter setting screen 475 may allow a user to additionally set ranges for normal and abnormal rangers, such as through upper and lower limits for normal ranges.

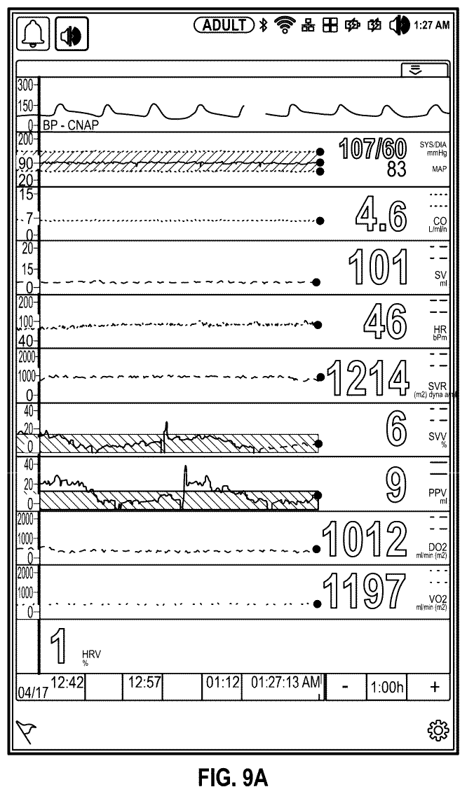

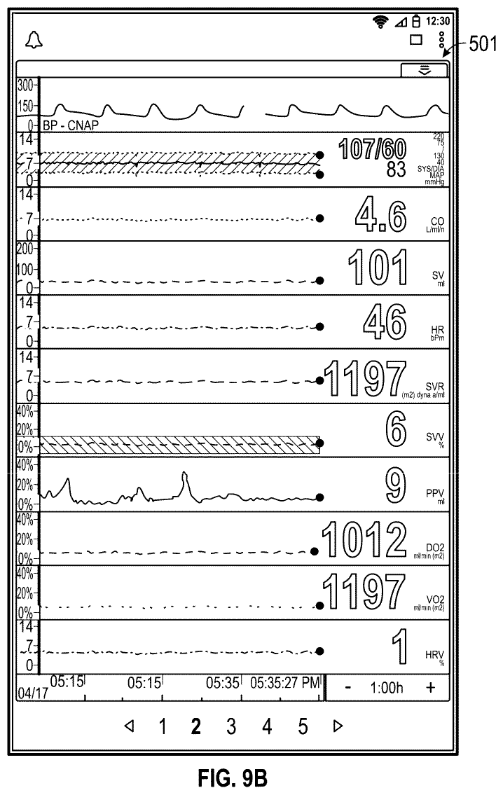

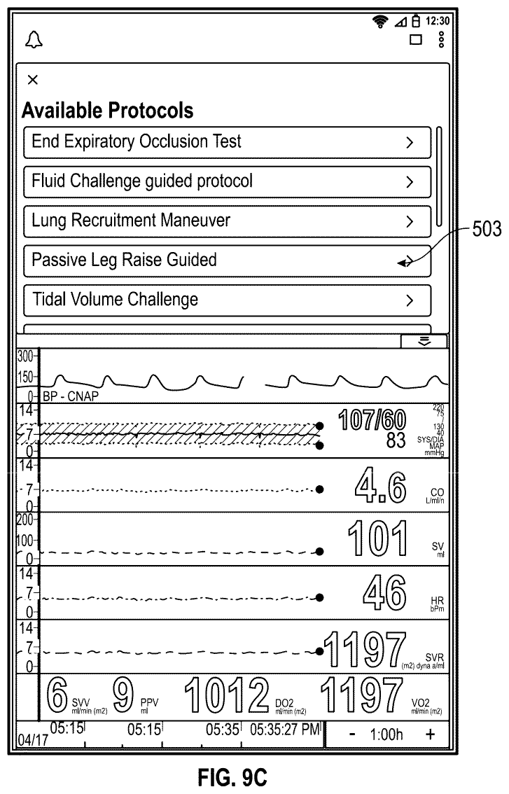

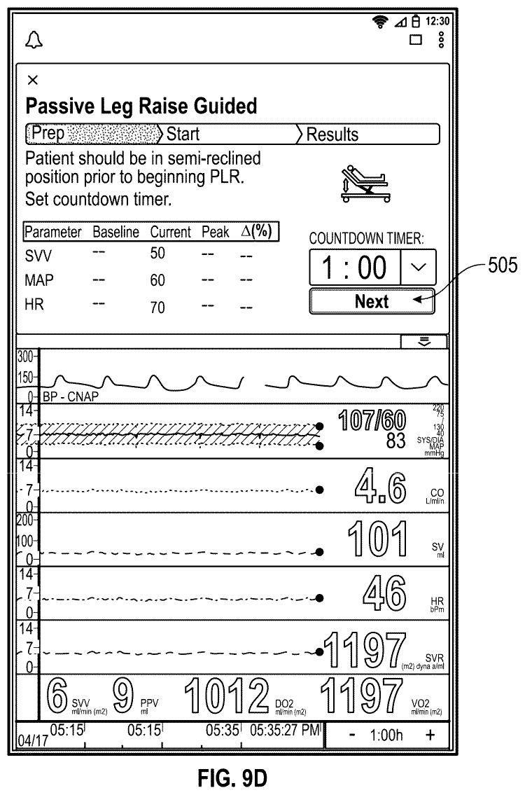

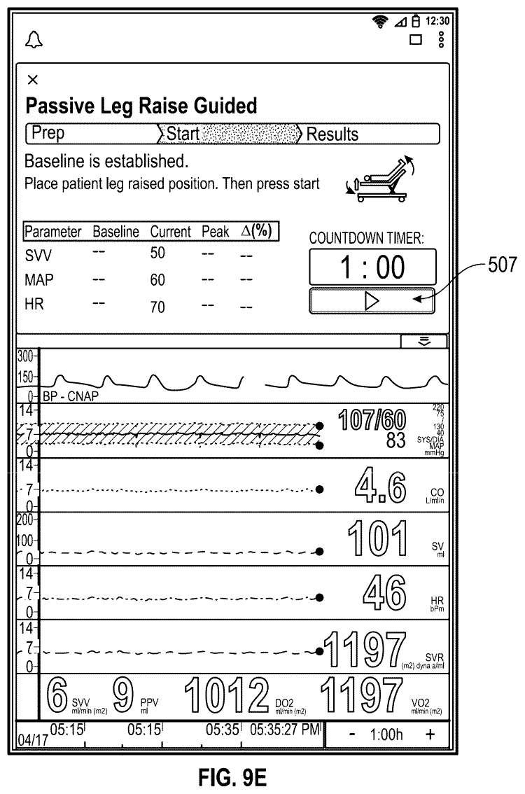

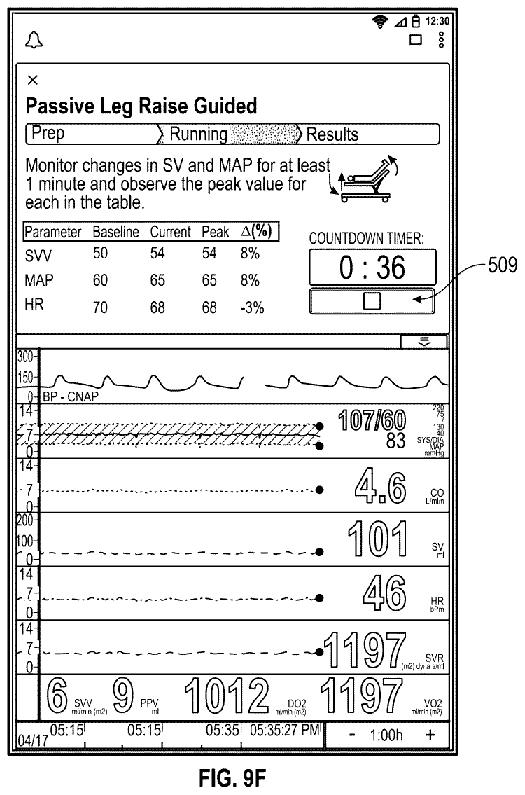

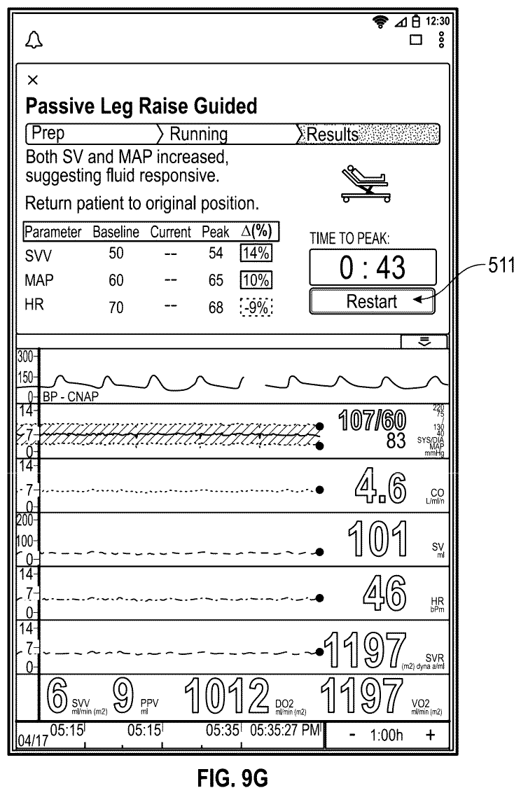

FIGS. 9A and 9B show example user interfaces that may display parameters or information relating to a patient's hemodynamic status and/or other physiological parameters or information. A user may select component 501 to navigate to a different user interface (such as shown in FIG. 9C) to select various physiological protocols.

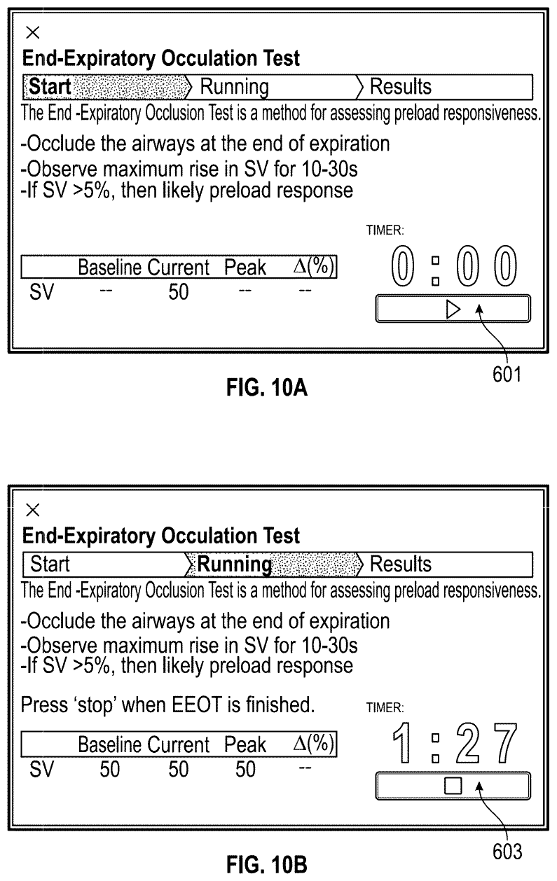

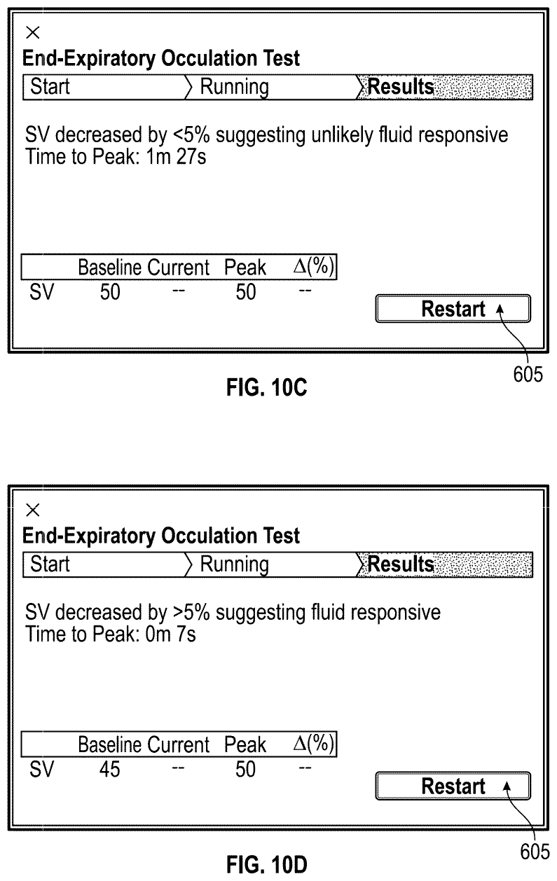

FIG. 9C shows an example user interface for selecting various physiological protocols. As shown the user interface may display a list of available protocols. In some embodiments, the user interface may display icons related to the protocols rather than a list. A user may select any of the displayed protocols to navigate to user interfaces relating to the selected protocol (for example, as shown and discussed with reference to FIGS. 10A-10D, 11A-11D, 12A-12C, 13A-13F). For example, a user may select component 503 to navigate to a “passive leg raise guided” protocol, as is shown and discussed with reference to FIGS. 9A-9G.

FIGS. 9D-9G show example user interfaces relating to a “passive leg raise guided” protocol. An example protocol may include systems and methods such as described with reference to U.S. Pat. No. 12,004,869, entitled “SYSTEM TO MONITOR AND MANAGE PATIENT HYDRATION VIA PLETHYSMOGRAPH VARIABLITY INDEX IN RESPONSE TO THE PASSIVE LEG RAISING,” filed on Nov. 4, 2019, the entire contents of which are hereby incorporated by reference herein in their entirety. As shown in FIG. 9D, a user may select component 505 to navigate to the user interface shown in FIG. 9E. As shown in FIG. 9E, a user may select component 507 to initiate the protocol and which may navigate the user to the user interface shown in FIG. 9F. As shown in FIG. 9F, a user may select component 509 to stop or pause the protocol and/or restart a stopped or paused protocol. As shown in FIG. 9G, the user interface may display the results of the protocol which may include a patient's hemodynamic status during and/or after the protocol. A user may select component 511 to navigate to the user interface shown in FIG. 9D or 9E to have the option to restart the protocol.

FIGS. 10A-10D, 11A-11D, 12A-12C, 13A-13F show additional example user interfaces relating to additional example protocols which may be displayed as an overlay of the user interfaces of FIG. 9A or 9B, as is shown with reference to the “passive leg raise guided” protocol user interfaces of FIGS. 9D-9G.

FIGS. 10A-10D show example user interfaces relating to a “end-expiratory occlusion test” protocol. A user may navigate to the user interface of FIG. 10A by selecting the corresponding protocol displayed in FIG. 9C, for example. As shown in FIG. 10A, a user may select component 601 to initiate the protocol and which may navigate the user to the user interface shown in FIG. 10B. As shown in FIG. 10B, a user may select component 603 to stop or pause the protocol and/or restart a stopped or paused protocol. As shown in FIG. 10C or 10D, the user interface may display the results of the protocol which may include a patient's hemodynamic status during and/or after the protocol. A user may select component 605 to navigate to the user interface shown in FIG. 10A to have the option to restart the protocol.

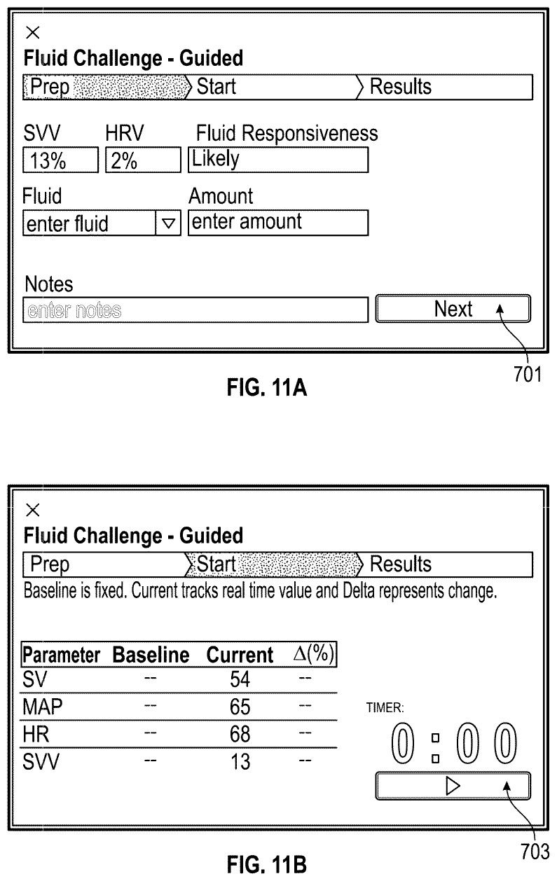

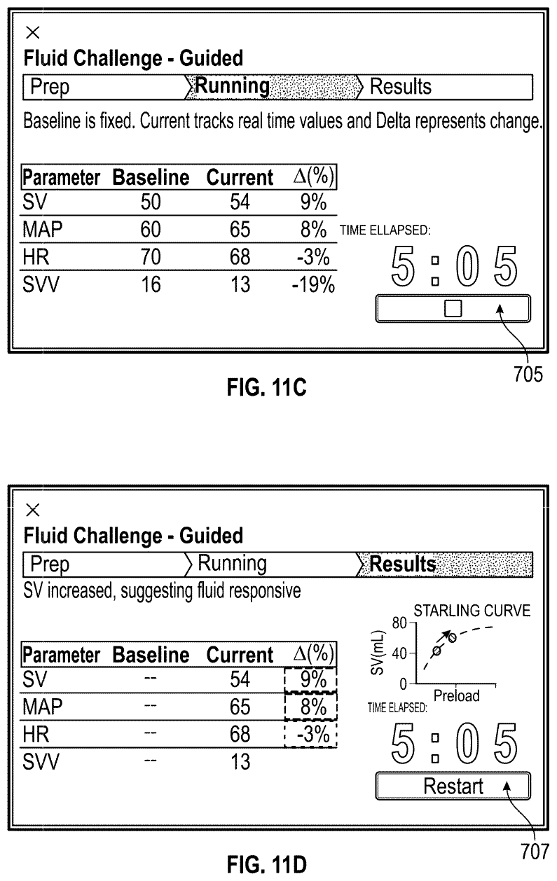

FIGS. 11A-11D show example user interfaces relating to a “fluid challenge-guided” protocol. A user may navigate to the user interface of FIG. 11A by selecting the corresponding protocol displayed in FIG. 9C, for example. As shown in FIG. 11A, a user may select component 701 to navigate to the user interface shown in FIG. 11B. As shown in FIG. 11B, a user may select component 703 to initiate the protocol and which may navigate the user to the user interface shown in FIG. 11C. As shown in FIG. 11C, a user may select component 705 to stop or pause the protocol and/or restart a stopped or paused protocol. As shown in FIG. 11D, the user interface may display the results of the protocol which may include a patient's hemodynamic status during and/or after the protocol. A user may select component 707 to navigate to the user interface shown in FIG. 11A or 11B to have the option to restart the protocol.

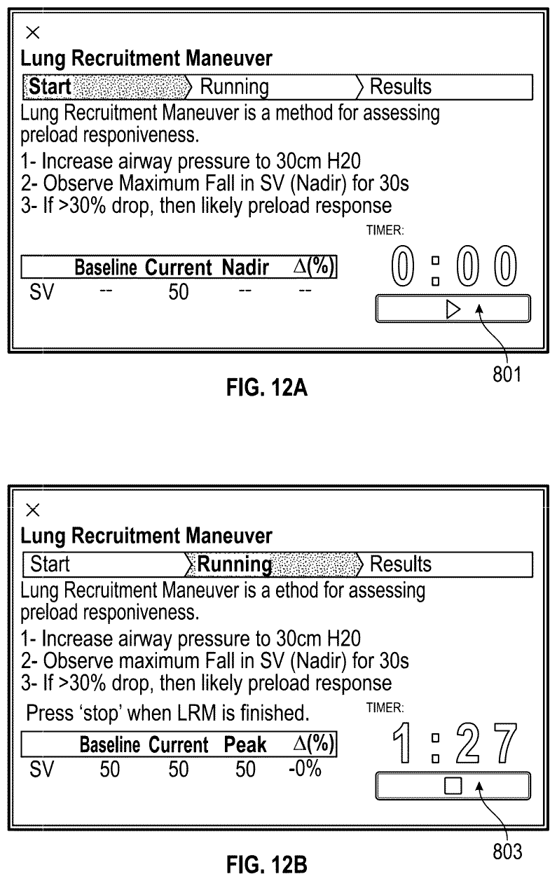

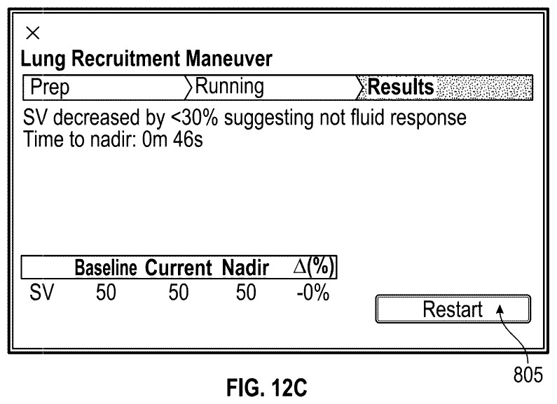

FIGS. 12A-12C show example user interfaces relating to a “lung recruitment maneuver” protocol. A user may navigate to the user interface of FIG. 12A by selecting the corresponding protocol displayed in FIG. 9C, for example. As shown in FIG. 12A, a user may select component 701 to initiate the protocol and which may navigate the user to the user interface shown in FIG. 12B. As shown in FIG. 12B, a user may select component 803 to stop or pause the protocol and/or restart a stopped or paused protocol. As shown in FIG. 12C, the user interface may display the results of the protocol which may include a patient's hemodynamic status during and/or after the protocol. A user may select component 805 to navigate to the user interface shown in FIG. 12A to have the option to restart the protocol.

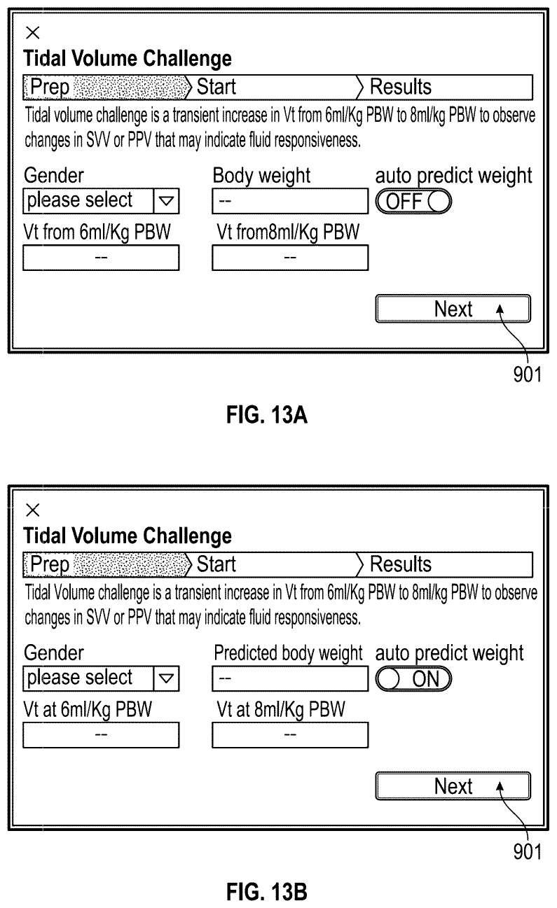

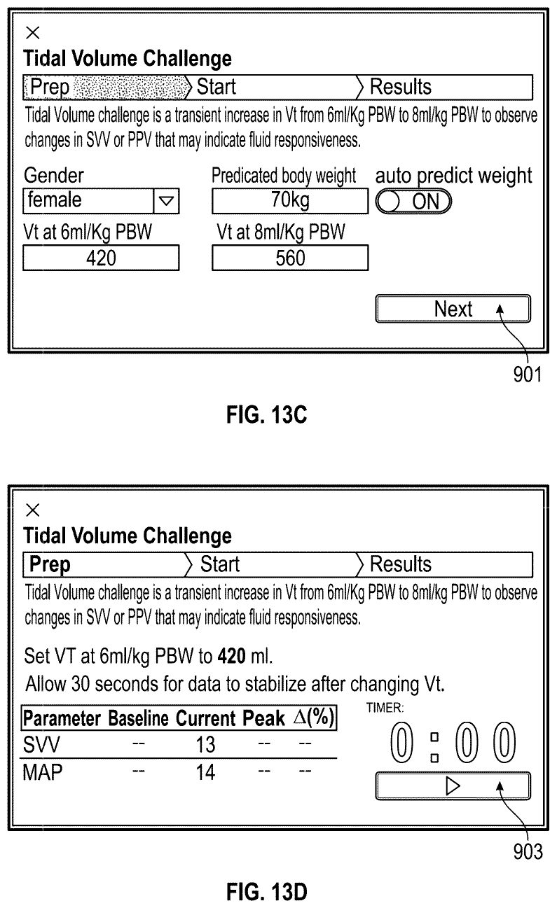

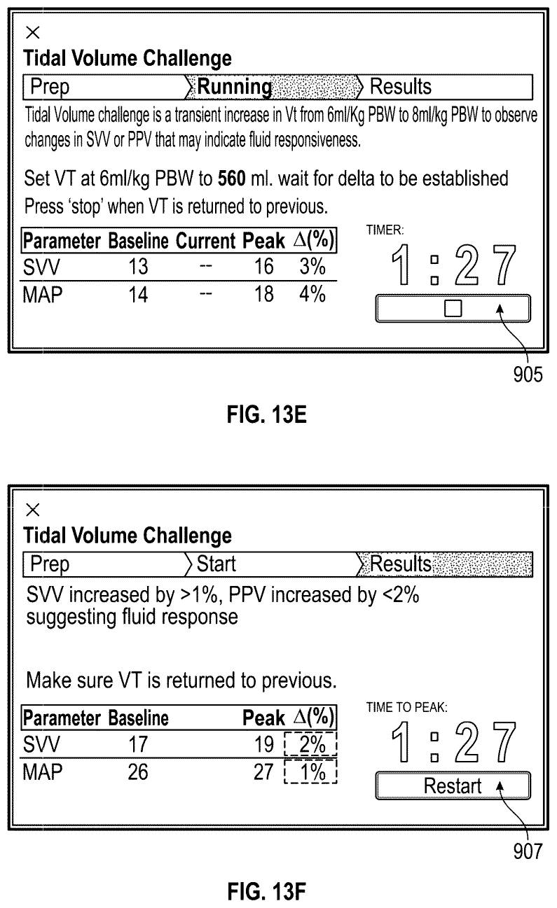

FIGS. 13A-13F show example user interfaces relating to a “tidal volume challenge” protocol. A user may navigate to the user interface of FIG. 13A or 13B or 13C by selecting the corresponding protocol displayed in FIG. 9C, for example. As shown in FIG. 13A or 13B a user may not be able to select component 901 to initiate the protocol. As shown in FIG. 13C, a user may be able to select component 901 to navigate to the user interface shown in FIG. 13D. As shown in FIG. 13D, a user may select component 903 to initiate the protocol and which may navigate the user to the user interface shown in FIG. 9E. As shown in FIG. 9E, a user may select component 905 to stop or pause the protocol and/or restart a stopped or paused protocol. As shown in FIG. 13F, the user interface may display the results of the protocol which may include a patient's hemodynamic status during and/or after the protocol. A user may select component 907 to navigate to one of the user interfaces shown in one of FIG. 13A, 13B, 13C or 13D to have the option to restart the protocol.

### Example Computing Environment for a Data Set Management System

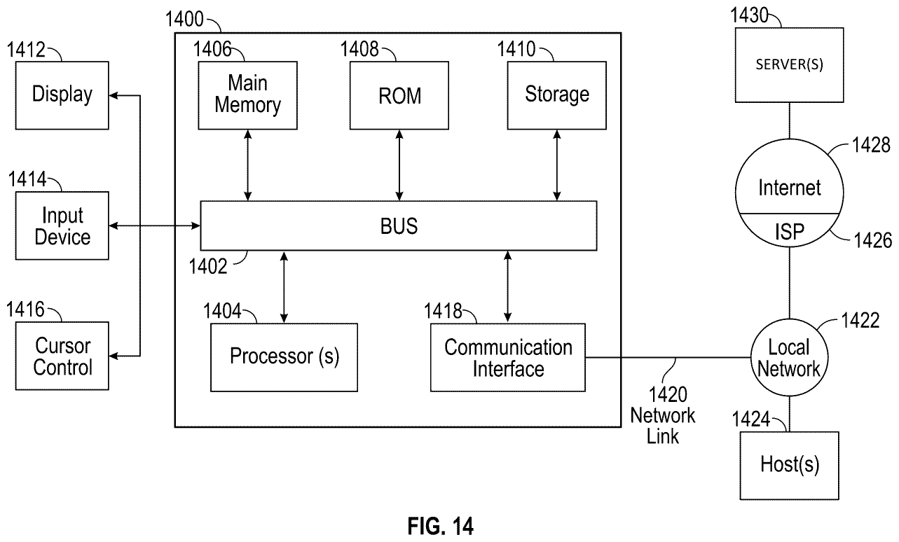

In an implementation, the system (e.g., one or more aspects of the system 100 and/or the like) may include, or be implemented in, a “virtual computing environment”. As used herein, the term “virtual computing environment” should be construed broadly to include, for example, computer-readable program instructions executed by one or more processors (e.g., as described in the example of FIG. 14) to implement one or more aspects of the modules and/or functionality described herein. Further, in this implementation, one or more services/modules/engines and/or the like of the system may be understood as comprising one or more rules engines of the virtual computing environment that, in response to inputs received by the virtual computing environment, execute rules and/or other program instructions to modify operation of the virtual computing environment. For example, a request received from a user computing device may be understood as modifying operation of the virtual computing environment to cause the request access to a resource from the system. Such functionality may include a modification of the operation of the virtual computing environment in response to inputs and according to various rules. Other functionality implemented by the virtual computing environment (as described throughout this disclosure) may further include modifications of the operation of the virtual computing environment, for example, the operation of the virtual computing environment may change depending on the information gathered by the system. Initial operation of the virtual computing environment may be understood as an establishment of the virtual computing environment. In some implementations the virtual computing environment may include one or more virtual machines, containers, and/or other types of emulations of computing systems or environments. In some implementations the virtual computing environment may include a hosted computing environment that includes a collection of physical computing resources that may be remotely accessible and may be rapidly provisioned as needed (commonly referred to as “cloud” computing environment).

Implementing one or more aspects of the system as a virtual computing environment may advantageously enable executing different aspects or modules of the system on different computing devices or processors, which may increase the scalability of the system. Implementing one or more aspects of the system as a virtual computing environment may further advantageously enable sandboxing various aspects, data, or services/modules of the system from one another, which may increase security of the system by preventing, e.g., malicious intrusion into the system from spreading. Implementing one or more aspects of the system as a virtual computing environment may further advantageously enable parallel execution of various aspects or modules of the system, which may increase the scalability of the system. Implementing one or more aspects of the system as a virtual computing environment may further advantageously enable rapid provisioning (or de-provisioning) of computing resources to the system, which may increase scalability of the system by, e.g., expanding computing resources available to the system or duplicating operation of the system on multiple computing resources. For example, the system may be used by thousands, hundreds of thousands, or even millions of users simultaneously, and many megabytes, gigabytes, or terabytes (or more) of data may be transferred or processed by the system, and scalability of the system may enable such operation in an efficient and/or uninterrupted manner.

Various implementations of the present disclosure may be a system, a method, and/or a computer program product at any possible technical detail level of integration. The computer program product may include a computer-readable storage medium (or mediums) having computer-readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer-readable storage medium (or mediums). Computer-readable storage mediums may also be referred to herein as computer-readable storage or computer-readable storage devices.

The computer-readable storage medium can include a tangible device that can retain and store data and/or instructions for use by an instruction execution device. The computer-readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may include copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions (as also referred to herein as, for example, “code,” “instructions,” “module,” “application,” “software application,” “service,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer-readable program instructions may be callable from other instructions or from itself, and/or may be invoked in response to detected events or interrupts. Computer-readable program instructions configured for execution on computing devices may be provided on a computer-readable storage medium, and/or as a digital download (and may be originally stored in a compressed or installable format that requires installation, decompression, or decryption prior to execution) that may then be stored on a computer-readable storage medium. Such computer-readable program instructions may be stored, partially or fully, on a memory device (e.g., a computer-readable storage medium) of the executing computing device, for execution by the computing device. The computer-readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to implementations of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart(s) and/or block diagram(s) block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowcharts and block diagrams in the Figures illustrate the architecture, functionality, and operation of possible implementations of systems, methods, and computer program products according to various implementations of the present disclosure. In this regard, each block in the flowchart or block diagrams may represent a service, module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted or optional in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, and/or the like with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above implementations may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, and/or the like), Windows CE, Unix, Linux, SunOS, Solaris, Blackberry OS, Vx Works, or other suitable operating systems. In other implementations, the computing devices may be controlled by a proprietary operating system. Conventional operating systems control and schedule computer processes for execution, perform memory management, provide file system, networking, I/O services, and provide a user interface functionality, such as a GUI, among other things.

For example, FIG. 14 shows a block diagram that illustrates a computer system 500 upon which various implementations and/or aspects (e.g., one or more aspects of the system 100, 200 and/or the like) may be implemented. Multiple such computer systems 1400 may be used in various implementations of the present disclosure. Computer system 1400 includes a bus 1402 or other communication mechanism for communicating information, and a hardware processor, or multiple processors, 1404 coupled with bus 1402 for processing information. Hardware processor(s) 1404 may be, for example, one or more general purpose microprocessors.

Computer system 1400 also includes a main memory 1406, such as a random-access memory (RAM), cache and/or other dynamic storage devices, coupled to bus 1402 for storing information and instructions to be executed by processor 1404. Main memory 1406 also may be used for storing temporary variables or other intermediate information during execution of instructions to be executed by processor 1404. Such instructions, when stored in storage media accessible to processor 1404, render computer system 1400 into a special-purpose machine that is customized to perform the operations specified in the instructions. The main memory 1406 may, for example, include instructions to implement server instances, queuing modules, memory queues, storage queues, user interfaces, and/or other aspects of functionality of the present disclosure, according to various implementations.

Computer system 1400 further includes a read only memory (ROM) 1408 or other static storage device coupled to bus 1402 for storing static information and instructions for processor 1404. A storage device 1410, such as a magnetic disk, optical disk, or USB thumb drive (Flash drive), and/or the like, is provided and coupled to bus 1402 for storing information and instructions.

Computer system 1400 may be coupled via bus 1402 to a display 1412, such as a cathode ray tube (CRT) or LCD display (or touch screen), for displaying information to a computer user. An input device 1414, including alphanumeric and other keys, is coupled to bus 1402 for communicating information and command selections to processor 1404. Another type of user input device is cursor control 1416, such as a mouse, a trackball, or cursor direction keys for communicating direction information and command selections to processor 1404 and for controlling cursor movement on display 1412. This input device typically has two degrees of freedom in two axes, a first axis (e.g., x) and a second axis (e.g., y), that allows the device to specify positions in a plane. In some implementations, the same direction information and command selections as cursor control may be implemented via receiving touches on a touch screen without a cursor.

Computing system 1400 may include a user interface module to implement a GUI that may be stored in a mass storage device as computer executable program instructions that are executed by the computing device(s). Computer system 1400 may further, as described below, implement the techniques described herein using customized hard-wired logic, one or more ASICs or FPGAs, firmware, and/or program logic which in combination with the computer system causes or programs computer system 1400 to be a special-purpose machine. According to one implementation, the techniques herein are performed by computer system 1400 in response to processor(s) 1404 executing one or more sequences of one or more computer-readable program instructions contained in main memory 1406. Such instructions may be read into main memory 1406 from another storage medium, such as storage device 1410. Execution of the sequences of instructions contained in main memory 1406 causes processor(s) 1404 to perform the process steps described herein. In alternative implementations, hard-wired circuitry may be used in place of or in combination with software instructions.

Various forms of computer-readable storage media may be involved in carrying one or more sequences of one or more computer-readable program instructions to processor 1404 for execution. For example, the instructions may initially be carried on a magnetic disk or solid-state drive of a remote computer. The remote computer can load the instructions into its dynamic memory and send the instructions over a telephone line using a modem. A modem local to computer system 1400 can receive the data on the telephone line and use an infra-red transmitter to convert the data to an infra-red signal. An infra-red detector can receive the data carried in the infra-red signal and appropriate circuitry can place the data on bus 1402. Bus 1402 carries the data to main memory 1406, from which processor 1404 retrieves and executes the instructions. The instructions received by main memory 1406 may optionally be stored on storage device 1410 either before or after execution by processor 1404.

Computer system 1400 also includes a communication interface 1418 coupled to bus 1402. Communication interface 1418 provides a two-way data communication coupling to a network link 1420 that is connected to a local network 1422. For example, communication interface 1418 may be an integrated services digital network (ISDN) card, cable modem, satellite modem, or a modem to provide a data communication connection to a corresponding type of telephone line. As another example, communication interface 1418 may be a local area network (LAN) card to provide a data communication connection to a compatible LAN (or WAN component to communicated with a WAN). Wireless links may also be implemented. In any such implementation, communication interface 1418 sends and receives electrical, electromagnetic, or optical signals that carry digital data streams representing various types of information.

Network link 1420 typically provides data communication through one or more networks to other data devices. For example, network link 1420 may provide a connection through local network 1422 to a host computer 1424 or to data equipment operated by an Internet Service Provider (ISP) 1426. ISP 1426 in turn provides data communication services through the worldwide packet data communication network now commonly referred to as the “Internet” 1428. Local network 1422 and Internet 1428 both use electrical, electromagnetic, or optical signals that carry digital data streams. The signals through the various networks and the signals on network link 1420 and through communication interface 1418, which carry the digital data to and from computer system 1400, are example forms of transmission media.

Computer system 1400 can send messages and receive data, including program code, through the network(s), network link 1420 and communication interface 1418. In the Internet example, a server 1430 might transmit a requested code for an application program through Internet 1428, ISP 1426, local network 1422 and communication interface 1418.

The received code may be executed by processor 1404 as it is received, and/or stored in storage device 1410, or other non-volatile storage for later execution.

As described above, in various implementations certain functionality may be accessible by a user through a web-based viewer (such as a web browser), or other suitable software program). In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain implementations, one or more of the computing devices and/or systems of the disclosure may include mobile computing devices, and user interfaces may be accessible through such mobile computing devices (for example, smartphones and/or tablets).

Many variations and modifications may be made to the above-described implementations, the elements of which are to be understood as being among other acceptable examples. All such modifications and variations are intended to be included herein within the scope of this disclosure. The foregoing description details certain implementations. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain implementations include, while other implementations do not include, certain features, elements, and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or disjunctive language such as the phrase “at least one of X, Y, or Z,” unless specifically stated otherwise, is to be understood with the context as used in general to convey that an item, term, and/or the like may be either X, Y, or Z, or any combination thereof (for example, X, Y, and/or Z). For example, the term “or” is used in its inclusive sense (and not in its exclusive sense) so that when used, for example, to connect a list of elements, the term “or” means one, some, or all of the elements in the list. Thus, such conjunctive or disjunctive language is not generally intended to imply that certain implementations require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a general-purpose computer comprising one or more processors should not be interpreted as excluding other computer components, and may possibly include such components as memory, input/output devices, and/or network interfaces, among others.

While the above detailed description has shown, described, and pointed out novel features as applied to various implementations, it may be understood that various omissions, substitutions, and changes in the form and details of the devices or processes illustrated may be made without departing from the spirit of the disclosure. As may be recognized, certain implementations of the inventions described herein may be embodied within a form that does not provide all of the features and benefits set forth herein, as some features may be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A system for monitoring a hemodynamic status of a patient, the system comprising:
a transducer configured to couple to the patient and measure a hemodynamic status of the patient;
a holder configured to couple the transducer to a mounting plate, the holder comprising:
a first movable arm configured to engage a first bracket arm of the mounting plate, the first movable arm including a first securing device extending from the first movable arm; and
an adapter in communication with the transducer and a display and comprising one or more hardware processors, wherein the one or more hardware processors are configured to:
generate one or more signals in response to the measurement of the transducer;
process the one or more signals to generate one or more physiological parameters;
generate, based at least in part on the one or more signals or the one or more physiological parameters, user interface data for rendering a graphical user interface without additional signal processing by a monitor;
transmit, to the monitor, the user interface data;
transmit, to the monitor, the one or more signals or the one or more physiological parameters;
receive, from the monitor, user input; and
execute instructions to control an operation of the adapter or transducer or monitor, according to the user input.

2. The system of claim 1, the system further comprising a second movable arm configured to engage a second bracket arm of the mounting plate, the second movable arm including a second securing device extending from the second movable arm.

3. The system of claim 1, wherein the hemodynamic status comprises a cardiac output of the patient.

4. The system of claim 1, wherein the transducer uses a lithium chloride indicator method to measure the hemodynamic status of the patient.

5. The system of claim 1, wherein the one or more hardware processors are configured to not transmit, to the monitor, the one or more signals, and to transmit, to the monitor, the one or more physiological parameters.

6. The system of claim 1, wherein the adapter comprises a button configured to cause the one or more hardware processors to alter a value of the one or more physiological parameters to an altered value.

7. The system of claim 6, wherein the altered value comprises a default value or zero value.

8. The system of claim 2, wherein the first bracket arm is separated from the second bracket arm by a width.

9. The system of claim 8, wherein the width comprises a distance between and including 26 mm and 30 mm.

10. The system of claim 1, the system further comprising the monitor, the monitor comprising the display and configured to display graphical user interfaces via the display and receive user input via the display.

11. A system for monitoring a hemodynamic status of a patient, the system comprising:
a holder configured to couple a transducer to a mounting plate, wherein the transducer is configured to couple to a patient and measure a hemodynamic status of the patient, the holder comprising:
a first movable arm configured to engage a first bracket arm of the mounting plate, the first movable arm including a first securing device extending from the first movable arm; and
a second movable arm configured to engage a second bracket arm of the mounting plate, the second movable arm including a second securing device extending from the second movable arm; and
wherein the holder is configured to secure to a plurality of mounting plates with varying widths.

12. The system of claim 11, wherein the transducer is configured to communicate with a patient monitor through an adapter.

13. The system of claim 11, wherein the holder is configured to secure to a plurality of mounting plates with varying depths of the first bracket arm and the second bracket arm.

14. The system of claim 11, wherein the first movable arm or the second movable arm is configured to be depressed inwards towards the transducer.

15. The system of claim 11, wherein the first movable arm or the second movable arm is configured to provide an outward force towards the first bracket arm or the second bracket arm when depressed inwards.

16. The system of claim 11, wherein the first movable arm or the second movable arm are integrated into the holder.

17. The system of claim 11, wherein the first securing device or the second securing device is integrated into the holder.

18. The system of claim 11, wherein the first securing device or the second securing device is configured to be depressed inwards towards the transducer.

19. The system of claim 11, wherein the first securing device or the second securing device is configured to provide an outward force towards the first bracket arm or the second bracket arm when depressed inwards.

20. The system of claim 11, wherein the holder is configured to couple the transducer to the mounting plate without using any adhesive.
